(19) United States
(12) Patent Application Publication
(10) Pub. No.: US 2024/0076375 A1
(43) Pub. Date: Mar. 7, 2024

(54) Anti-TIM-3 Antibodies and Use Thereof

(71) Applicant: BeiGene Switzerland GmbH, Basel (CH)

(72) Inventors: Tong Zhang, Beijing (CN); Liu Xue, Beijing (CN); Qi Liu, Beijing (CN); Hao Peng, Beijing (CN); Min Wei, Beijing (CN); Kang Li, Beijing (CN)

(21) Appl. No.: 18/475,552

(22) Filed: Sep. 27, 2023

Related U.S. Application Data
(63) Continuation of application No. 17/526,817, filed on Nov. 15, 2021, now abandoned.
Application No. 17/526,817 is a continuation of application No. 16/328,047, filed on Feb. 25, 2019, now Pat. No. 11,203,637.

(30) Foreign Application Priority Data
Aug. 26, 2016 (WO) PCT/CN2016/096924

(51) Int. Cl. C07K 16/28; A61K 39/395; A61P 35/00; A61P 37/00

(52) U.S. Cl. CPC C07K 16/2803; A61K 39/395; A61P 35/00; A61P 37/00; C07K 16/2818; A61K 2039/507

(57) ABSTRACT

Provided are antibodies that specifically bind to T-cell immunoglobulin domain and mucin domain 3 (Tim-3). The anti-Tim-3 antibodies can be used to treat, prevent or diagnose immune, cancerous, infectious diseases or other pathological disorders that may be modulated by Tim-3-mediated functions.

### CROSS-REFERENCE TO RELATED APPLICATIONS

This application is a continuation of U.S. patent application Ser. No. 17/526,817, filed Nov. 15, 2021, which is a continuation of U.S. patent application Ser. No. 16/328,047, filed Feb. 25, 2019, now U.S. Pat. No. 11,203,637 B2 issued on Dec. 21, 2021, which is the U.S. national stage application to International Patent Application No. PCT/CN2017/099098, filed Aug. 25, 2017, which claims the benefit of priority to International Application No. PCT/CN2016/096924 filed on Aug. 26, 2016, the entire contents of each of which are incorporated herein by reference.

### DESCRIPTION OF THE TEXT FILE SUBMITTED ELECTRONICALLY

This application contains a sequence listing, which is submitted electronically. The contents of the electronic sequence listing (065829.89US3 Sequence Listing.xml; Size: 63,196 bytes; and Date of Creation: Sep. 27, 2023) is herein incorporated by reference in its entirety.

### FIELD OF THE INVENTION

Disclosed herein are antibodies that specifically bind to T-cell immunoglobulin domain and mucin domain 3 (Tim-3).

### BACKGROUND OF THE INVENTION

T-cell immunoglobulin domain and mucin domain 3 (Tim-3, HAVCR2, or CD366) is a 33 KD type I transmembrane glycoprotein, a member of the T-cell Immunoglobulin- and mucin-domain-containing family that plays an important role in promoting I-cell exhaustion in both chronic viral infections and tumor escape from immune surveillance (Monney et al., 2002 Nature 415:536-541; Sanchez-Fueyo A, et al., 2003 Nat Immunol. 4:1093-101; Sabatos C A, et al., 2003 Nat Immunol. 4:1102-10; Anderson et al., 2006 Curr Opin Immunol 18:665-669). The genes and cDNAs coding for Tim-3 were cloned and characterized in mouse and human (Monney et al., 2002 Nature 415:536-541; McIntire et al., 2001 Nat. Immunol. 2:1109-1116). Mature human Tim-3 contains 280 amino acid residues (NCBI accession number: NP_116171.3). Its extracellular domain consists of amino acid residues 1-181, and the transmembrane domain and cytoplasmic C-terminal tail comprises residues 182-280. There are no known inhibitory signaling motifs, such as immunoreceptor tyrosine-based inhibitory motif (ITIM) and tyrosine switch motif (ITSM), found in the cytoplasmic domain.

Tim-3 was initially identified in Th1 cells. Subsequent studies showed that in addition to T cells, Tim-3 was also expressed in other types of immune cells, such as NK cells, macrophages, DCs, and mast cells (Hastings et al., 2009 Eur J Immunol 39:2492-2501; Anderson et al., 2007 Science 318: 1141-1143; Phong B L, et al., 2015 J Exp Med. pii: jem. 20150388). Tim-3 is rarely expressed in other human tissues. In T cells, Tim-3 expression is positively regulated through TCR/CD3 activation (Hastings et al., 2009 Eur J Immunol 39:2492-2501). In addition, common γ chain cytokines (e.g., IL-2, IL-7, IL-15 and IL-21) also increase Tim-3 expression in a PI-3 kinase-dependent manner (Mujib S. et al., 2012 J Immunol. 188:3745-56). T cells in tumor microenvironment (TME) often co-express Tim-3 with other “checkpoint” inhibitory immune receptors, such as PD-1, Lag-3 and Tigit (Fourcade J, et al., 2010 J Exp Med 207:2175-86; Gros A, et al., 2014 J Clin Invest. 124:2246-59).

Up to date, several Tim-3 ligands (Tim-3L) have been reported, which include galectin-9 and phosphatidylserine (PtdSer), being considered as two major ones (Anderson A C, 2012 Curr Opin Immunol. 24:213-6). Binding of Tim-3Ls to Tim-3 receptor induces intracellular signaling that inhibits T-cell activation, leading to diminished cell proliferation, IL-2 and IFN-γ secretion (Dekruyff et al., 2010 J. Immunol. 184:1918-1930; Zhu et al., 2005 Nat. Immunol. 6:1245-1252). The detailed mechanisms of Tim-3 signaling in T cells still remain largely unknown. Some studies have shown that Tim-3 could be recruited to the immunological synapses and sequester Src kinase Lck when interacting with TCR, whereby inhibiting its signaling, especially NFAT signaling pathway (Tomkowicz B, et al., 2015 PLoS One 10:e0140694; Clayton K L, et al., 2014 J. Immunol. 192:782-91).

In the cancer and viral infections, activation of Tim-3 signaling promotes immune cell dysfunction, leading to the cancer outgrowth or extended viral infection. Up-regulation of Tim-3 expression in tumor-infiltrating lymphocytes (TILs), macrophages and tumor cells has been reported in many types of cancers such as lung (Zhuang X, et al., Am J Clin Pathol 2012 137: 978-985), liver (Li H, et al., Hepatology 2012 56:1342-1351), stomach (Jiang et al., PLoS One 2013 8:e81799), kidney (Komohara et al., Cancer Immunol Res. 2015 3:999-1000), breast (Heon E K, et al., 2015 Biochem Biophys Res Commun. 464:360-6), colon (Xu et al., Oncotarget 2015), melanocytes (Gros A, et al., 2014 J Clin invest. 2014 124:2246-2259) and cervical cancer (Can et al., PLoS One 2013 8:e53834). The increased expression of Tim-3 in those cancers is associated with poor prognosis of patient survival outcome. Not only does up-regulation of Tim-3 signaling play important roles in immune tolerance to cancer, but also to chronic viral infection, Duffing HIV and HCV infections, expression of Tim-3 on T cells was significantly higher compared to that in healthy people and positively correleated with viral loads and disease progression (Jones R B, et al., 2008 J Exp Med. 205:2763-79; Sakhdari A. et al., 2012 PLoS One 7:e40146; Golden Mason L, et al., 2009 J Virol. 83:9122-30; 2012 Moorman P, et al., J Immunol. 189: 755-66). In addition, blockade of Tim-3 receptor alone or in combination with PD-1/PD-L1 blocakde could rescue functionally “exhausted” cells both in vitro and in vivo (Dietze K K, et al., 2013 PLoS Pathog 9:e1003798; Golden-Mason L, et al., 2009 J Virol 83:9122-30). Therefore, modulation of Tim-3 signaling by therapeutic agents may rescue immune cells, e.g., T cells, NK cells and macrophages (Mφ), from tolerance, inducing efficient immune responses to eradicate tumors or chronic viral infections.

It has been reported that some antibodies can be internalized upon binding to its target on the cell surface (Hurwitz E, et al., 1995, Proc Natl Acad Sci USA 92:3353-7; Poul M A, et al., 2000, J Mol Biol. 301:1149-61; Fischer E, et al., 2012, Clin Cancer Res. 18:6208-18). Antibody-induced receptor endocytosis leads to down-modulation of receptors on the cell surface and inhibition of receptor-dependent signaling (Liu L, et al., 2014. Clin Cancer Res. 20:6059-70). As antibody internalization will reduce surface expression of a receptor, a persistent internalization is usually desirable for an antibody of the receptor.

Therefore, there is a need of an anti-Tim-3 antibody which has a high affinity and specificity to Tim-3 receptor and preferably further has a persistent internalization of Tim-3 receptor.

### SUMMARY OF THE INVENTION

Disclosed herein are antibody molecules that bind to Tim-3 with high affinity and specificity. In particular, the anti-Tim-3 antibody disclosed herein provides a persistent or durable internalization of Tim-3 receptor. Also provided are nucleic molecules encoding the antibody molecules, expression vectors, host cells and methods for making the antibody molecules. Pharmaceutical compositions comprising the antibody molecules are also provided. The anti-Tim-3 antibody molecules disclosed herein can be used, alone or in combination with other agents or therapeutic modalities, to treat, prevent and/or diagnose diseases that are associated with suppression of immune cells by Tim-3-mediated intracellular signaling, e.g., immune disorders, cancer, infectious disease, Crohn's disease, sepsis, systemic inflammatory response syndrome (SIRS), and glomerulonephritis. Thus, compositions and methods for treating various disorders or diseases mentioned above using the anti-Tim-3 antibody molecules are disclosed herein, and use of the anti-Tim-3 antibody molecules in manufacturing medicine for treating various disorders or diseases mentioned above, are also provided.

In certain aspects, this disclosure provides an anti-Tim-3 antibody capable of binding to human Tim-3, which includes at least one, two, three, four, five, or six complementarity determining regions (CDR's) comprising an amino acid sequence of SEQ ID NOs 3-8, or 26-27 or variants thereof comprising one or more conservative substitutions.

In some embodiments, the anti-Tim-3 antibody includes at least one, two or three CDRs from a heavy chain variable region (VH) comprising an amino acid sequence of SEQ TD NOs 3-5 or 26 or variants thereof comprising one or more conservative substitutions. In some embodiments, the anti-Tim-3 antibody includes at least one, two or three CDRs from a light chain variable region (VL) comprising an amino acid sequence of SEQ NOs 6-8 or 27 or variants thereof comprising one or more conservative substitutions.

In some embodiments, the anti-Tim-3 antibody includes at least one, two, three, four, five or six CDRs from a heavy and light chain variable region comprising an amino acid sequence of SEQ ID NOs 3-8, or 26-27 or variants thereof comprising one or more conservative substitutions.

In some embodiments, the anti-Tim-3 antibody includes six CDRs from a heavy and light chain variable region comprising an amino acid sequence of SEQ ID NOs 3-8, or 26-27 or variants thereof comprising one or more conservative substitutions.

In some embodiments, the anti-Tim-3 antibody comprises:
(a) a heavy chain variable region (VH) comprising one, two or three CDR amino acid sequences selected form SEQ ID NOs 3, 4, 5, or 26 or variants thereof comprising one or more conservative substitutions; and/or
(b) a light chain variable region (VL) comprising one, two or three CDR amino acid sequences selected form SEQ ID NOs 6, 7, 8, or 27 or variants thereof comprising one or more conservative substitutions.

In some embodiments, the anti-Tim-3 antibody comprises:
(a) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SIE ID NO 7 or variants thereof comprising one or more conservative substitutions and a VL-CDR3 amino acid sequence of SEQ ID NO 8 or variants thereof comprising one or more conservative substitutions;
(b) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 26 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 8 or variants thereof comprising one or more conservative substitutions;
(c) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions; or
(d) a heavy chain variable region (VH) comprising a VH-CDR2 amino acid sequence of SEQ ID NO 3 or variants thereof comprising one or more conservative substitutions, a VH-CDR2 amino acid sequence of SEQ ID NO 26 or variants thereof comprising one or more conservative substitutions and a VH-CDR3 amino acid sequence of SEQ ID NO 5 or variants thereofcomprising one or more conservative substitutions; and a light chain variable region (VL) comprising a VL-CDR1 amino acid sequence of SEQ ID NO 6 or variants thereof comprising one or more conservative substitutions, a VE-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions.

In some embodiments, the anti-Tim-3 antibody comprises:
(a) a heavy chain variable region (VH) comprising a VH-CDR1 amino acid sequence of SEQ ID NO 3, a VH-CDR2 amino acid sequence of SEQ ID NO 4 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 8; or
(b) a heavy chain variable region (VH) comprising a VH-CDR1 amino acid sequence of SEQ ID NO 3, a VH-CDR2 amino acid sequence of SEQ ID NO 26 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 27.

In some embodiments, the anti-Tim-3 antibody is a humanized antibody molecule.

In some embodiments, the anti-Tim-3 antibody is a humanized monoclonal antibody (mAb) molecule.

In some embodiments, the antibody comprises a heavy chain variable domain having at least 95%, 96%, 97%, 98%, 99% or 100% sequence identity with the amino acid sequence of SEQ ID NOs 9, 17, 28 or 40. In some embodiments, the antibody comprises a heavy chain variable domain having at least 95%, 96%, 97%, 98%, 99% or 100% sequence identity with the amino acid sequence of SEQ ID NOs 9, 17 or 28. In some embodiments, the anti-Tirn-3 antibody comprises a light chain variable domain having at least 95%, 96%, 97%, 98%, 99% or 100% sequence identity with the amino sequence of SEQ ID NOs 11, 19, 30 or 36.

In some embodiments, the anti-Tim-3 antibody comprises:
(a) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 11;
(b) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 19;
(c) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 30;
(d) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 36;
(e) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 11;
(f) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 19;
(g) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 30;
(h) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 36;
(i) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 11;
(j) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 19;
(k) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 30;
(l) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 36;
(m) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 11;
(n) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 19;
(o) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 30; or
(p) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 36.

In some embodiments, the anti-Tim-3 antibody comprises:
(a) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 11;
(b) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 19;
(c) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 30;
(d) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 36;
(e) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 11;
(f) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 19;
(g) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 30;
(h) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 36;
(i) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 11;
(j) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 19;
(k) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 30; or;
(l) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 36.

In some embodiments, the anti-Tim-3 antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30;
(d) a heavy chain variable domain comprising the amino acid sequence of SEQ ID No 28, and a light chain variable domain comprising the amino acid sequence of SEQ ID NO 36;
(e) a heavy chain variable domain comprising the amino acid sequence of SEQ ID NO 40, and a light chain variable domain comprising the amino acid sequence of SEQ ID NO 36.

In some embodiments, the anti-Tim-3 antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30; or
(d) a heavy chain variable domain comprising the amino acid sequence of SEQ ID NO 28, and a light chain variable domain comprising the amino acid sequence of SEQ ID NO 36.

In some embodiments, the anti-Tim-3 antibody comprises a heavy chain having at least 95%, 96%, 97%, 98%, 99% or 100% sequence identity with the amino acid sequence of SEQ ID NO 13, 22 or 32. In some embodiments, the anti-Tim-3 antibody comprises a light chain having at least 95%, 96%, 97%, 98%, 99% or 100% sequence identity with the amino acid sequence of SEQ ID NO 15, 24, 34 or 38.

In some embodiments, the anti-Tim-3 antibody comprises:
(a) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 15;
(b) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 24;
(c) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 34;
(d) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 38;
(e) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 15;
(f) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 24;
(g) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 34;
(h) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 38;
(i) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 15;
(j) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 24;
(k) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 34; or
(l) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 38.

In some embodiments, the anti-Tim-3 antibody comprises:
(a) a heavy chain comprising the amino acid sequence of SEQ ID NO 13, and a light chain comprising the amino acid sequence of SEQ ID NO 15;
(b) a heavy chain comprising the amino acid sequence of SEQ ID NO 22, and a light chain comprising the amino acid sequence of SEQ ID NO 24;
(c) a heavy chain comprising the amino acid sequence of SEQ ID NO 32, and a light chain comprising the amino acid sequence of SEQ ID NO 34; or
(d) a heavy chain comprising the amino acid sequence of SEQ ID NO 32, and al light chain comprising the amino acid sequence of SEQ ID NO 38.

In some embodiments, the anti-Tim-3 antibody comprises one or more of:
(a) a light chain with an Aspartic acid to Glutamic acid mutation at position 1 of SEQ ID NO 24;
(b) a light chain with a Leucine to Methionine mutation at position 4 of SEQ ID NO 24;
(c) a light chain with a Valine to Isoleucine mutation at position 62 of SEQ ID NO 24;
(d) a light chain with a Aspartic acid to Glutamic acid mutation at position 74 of SEQ ID NO 24;
(e) a light chain with a Methionine to Leucine mutation at position 96 of SEQ ID NO 24;
(f) a heavy chain with a Phenylalanine to Tyrosine mutation at position 59 of SEQ ID NO 22;
(g) a heavy chain with a Proline to Valine mutation at position 60 of SEQ ID NO 22;
(h) a heavy chain with a Serine to Threonine mutation at position 77 of SEQ ID NO 22; or
(i) a heavy chain with a Cysteine to Leucine mutation at position 78 of SEQ ID NO 22.

In some embodiments, the anti-Tim-3 antibody is a Fab, F(ab′)2, Fv, or a single chain Fv (Scfv).

In some embodiments, the anti-Tim-3 antibody comprises a heavy chain constant region of the subclass of IgG1, IgG2, IgG3, IgG4 or a variant thereof, and a light chain constant region of the type of kappa or lambda or a variant thereof.

In some embodiments, the anti-Tim-3 antibody comprises a variant human IgG1 heavy chain constant region comprising the amino acid sequence of SEQ ID NO 21, and a human kappa. light chain constant region.

In some embodiments, the anti-Tim-3 antibody is isolated or recombinant.

In certain aspects, the present disclosure provides a composition, e.g., a pharmaceutical composition, comprising at least one of the antibody molecules described herein, and a pharmaceutically acceptable excipient. In some embodiments, the pharmaceutical composition includes a combination of the anti-Tim-3 antibody and one or more other agents, e.g., a therapeutic agent or other antibody molecule. In some embodiments, the anti-Tim-3 antibody is conjugated to a label or a therapeutic agent.

In certain aspects, the present disclosure also provides a method of stimulating an immune response in a subject, The method comprises administrating to a subject an antibody described herein (e.g., a therapeutically effective amount of an anti-Tim-3 antibody molecule), alone or in combination with one or more agents or procedures.

In certain aspects, the present disclosure also provides a method for treating (e.g., one or more of reducing, inhibiting, or delaying progression of) a cancer or a tumor in a subject. The method comprises, administrating to a subject an antibody described herein (e.g., a therapeutically effective amount of an anti-Tim-3 antibody molecule), alone or in combination with one or more agents or procedures. In some embodiments, the anti-Tim-3 antibody is administrated in combination with a chemotherapy, a targeted therapy, an oncolytic drug, a cytotoxic agent, an immune-based therapy, a cytokine, a surgical procedure, a radiation procedure, an activator of a costimulatory molecule, an inhibitor of an inhibitory molecule, a vaccine, or a cellular immunotherapy. In some embodiments, the anti-Tim-3 antibody is administrated in combination with an inhibitor of an immune checkpoint molecule selected from PD-1, PD-L1, CTLA-4, LAG-3, CEACAM-1, CEACAM-5, VISTA, BTLA, LAIR1, CD160, 2B4 or TCER. In some embodiments, the anti-Tim-3 antibody is administrated in combination with an anti-PD-1 mAb 317-4B6 (also named Hu317-4B6, 317-4B6/IgG4mt10, described in U.S. Pat. No. 8,735,553).

In certain embodiments, the cancer includes, but is not limited to, a lung cancer, a liver cancer, a stomach cancer, a cervical cancer, a melanoma, a renal cancer, a breast cancer, a colorectal cancer, a leukemia, a lymphoma, an ovarian cancer, a head and neck cancer or a metastatic lesion of the cancer.

In further aspects, this disclosure provides a method of treating an infectious disease, comprising administering to a subject a therapeutically effective amount of an anti-Tim-3 antibody described herein, alone or in combination with one or more agents or procedures. In some embodiments, the infectious disease is a chronic viral infectious disease, selected from HIV infection and HCV infection.

In some aspects, the present disclosure also provides use of the anti-Tim-3 antibody molecules in manufacturing medicine for treating various disorders or diseases described herein.

The anti-Tim-3 antibody molecules described herein show a special set of effector functions and physicochemical properties, which can inhibit Tim-3-mediated cellular signaling in immune cells, re-activate immune cells and enhance immunity. And, the mAbs in the format of full-length human IgG1 with modified heavy chain constant region have a unique set of features in the aspects of effector functions. The anti-Tim-3 mAbs were also humanized with high degree of similarity to human antibody molecules. In addition, the anti-Tim-3 antibody can synergize with an anti-PD-1 antibody, to activate T cells in vitro, to reduce tumor growth. Thereby, the anti-Tim-3 antibodies disclosed here may have therapeutic utility in treatment of cancer, viral infections and other human diseases that are mechanistically associated with immune tolerance or “exhaustion”.

### BRIEF DESCRIPTION OF THE DRAWINGS

FIG. 1 shows schematic diagrams of Tim-3-mIgG2a (top) and Tim-3-huIgG1 (bottom), in which L is a linker, N is N-terminus and C is C-terminus.

FIGS. 2A-2B show the phylogenetic trees of anti-Tim-3 antibodies. FIG. 2A shows the phylogenetic tree of anti-Tim-3 antibody heavy chain variable (Vh) regions; FIG. 2B shows the phylogenetic tree of anti-Tim-3 antibody light chain variable (Vl) regions. A total of 23 Tim-3 Vh and 20 Vκ sequences were aligned using DNASTAR's Megalign software. Sequence homology was displayed in the phylogenetic trees.

FIG. 3 shows the comparison of Tim-3 binding affinities of hu425-2E-1 and hu425-2F-1 to that of hu425-1-1 in ELISA.

FIG. 4 shows the Tim-3 binding affinities of mAbs 1G5 and Ab2000 together with that of hu425-2-3b in ELISA.

FIG. 5 shows the inhibition of Tim-3-mediated phagocytosis by an anti-Tim-3 antibody hu425-2-3b.

FIG. 6 shows activation of IFN-γ secretion by anti-Tim-3 antibodies, including ch425 and hu425-1-1, in primary human PBMCs.

FIG. 7 shows activation of CMV-specific human T cells by anti-Tim-3 antibody hu425-2-3b.

FIGS. 8A-8B show anti-Tim-3 antibody hu425-2-3b promotes NK cell-mediated cytotoxicity.

FIG. 9 shows anti-Tim-3 antibody hu425-2-3b reduces the surface expression of Tim-3 receptor.

FIG. 10 shows internalization of Tim-3 receptor of anti-Tim-3 antibodies (hu425-2-3b, Ab1 and Ab2).

FIG. 11 shows anti-Tim-3 antibody hu425-2-3b, alone or in combination with an anti-PD-1 antibody hu317-4B6, enhances IFN-γ secretion in MLR.

FIGS. 12A-12B show anti-Tim-3 antibody hu425-2-3b did not induce ADCC (antibody-dependent cell-mediated cytotoxicity) and CDC (complement dependent cytotoxicity).

FIG. 13 shows the anti-tumor effects of anti-Tim-3 antibody ch425, anti-PD-1 antibody hu317-4B6, and combination thereof in a human A431 allogeneic xenograft model.

### DETAIL DESCRIPTION OF THE INVENTION

### Definitions

Exemplary Conservative Amino Acid Substitutions

| Original amino | One-letter and | Conservative |
|---|---|---|
| acid residue | three-letter codes | substitution |
| Alanine | A or Ala | Gly; Ser |
| Arginine | R or Arg | Lys; His |
| Asparagine | N or Asn | Gln; His |
| Aspartic acid | D or Asp | Gln; Asn |
| Cysteine | C or Cys | Ser; Ala |
| Glutamine | Q or Gln | Asn |
| Glutamic acid | E or Glu | Asp; Gln |
| Glycine | G or Gly | Ala |
| Histidine | H or His | Asn; Gln |
| Isoleucine | I or Ile | Leu; Val |
| Leucine | L or Leu | Ile; val |
| Lysine | K or Lys | Arg; His |
| Methionine | M or Met | Leu; Ile; Tyr |
| Phenylalanine | F or Phe | Tyr; Met; Leu |
| Proline | P or Pro | Ala |
| Serine | S or Ser | Thr |
| Threonine | T or Thr | Ser |
| Tryptophan | W or Trp | Tyr; Phe |
| Tyrosine | Y or Tyr | Trp; Phe |
| Valine | V or Val | Ile; Leu |

Unless specifically defined elsewhere in this document, all other technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

Throughout this specification and the claims which follow, unless the context requires otherwise, the word “eomprise”, and variations such as “comprises” and “comprising”, will be understood to imply the inclusion of a stated amino acid sequence, DNA sequence, step or group thereof, but not the exclusion of any other amino acid sequence, DNA sequence, step. When used herein the term “comprising” can be substituted with the term “containing”, “including” or sometimes “having”.

The term “Tim-3” includes various mammalian isoforms, e.g., human Tim-3, species homologs of human Tim-3, and analogs comprising at least one epitope within Tim-3. And the amino acid sequence of Tim-3, e.g., human Tim-3, and the nucleotide sequence encoding the same, is known in the art.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

### Antibody or Antibody Molecule

Disclosed herein are antibody molecules that bind to Tim-3 with high affinity and specificity.

In some embodiments, the anti-Tim-3 antibody includes at least one, two, three, four, five or six complementarity determining regions (CDR's) comprising an amino acid sequence SEQ ID Os 3-8, or 26-27 or variants thereof comprising one or more conservative substitutions.

In some embodiments, the anti-Tim-3 antibody includes at least one, two or three CDRs from a heavy chain variable region (VH) comprising an amino acid sequence of SEQ ID NOs 3-5 or 26 or variants thereof comprising one or more conservative substitutions. In some embodiments, the anti-Tim-3 antibody includes at least one, two or three CDRs from a light chain variable region (VL) comprising an amino acid sequence of SEQ NOs 6-8 or 27 or variants thereof comprising one or more conservative substitutions.

In some embodiments, the anti-Tim-3 antibody includes at least one, two, three, four, five or six CDRs from a heavy and light chain variable region comprising an amino acid sequence of SEQ ID NOs 3-8, or 26-27 or variants thereof comprising one or more conservative substitutions.

In some embodiments, the anti-Tim-3 antibody includes six CDRs from a heavy and light chain variable region comprising an amino acid sequence of SEQ ID NOs 3-8, or 26-27 or variants thereof comprising one or more conservative substitutions.

In some embodiments, the anti-Tim-3 antibody comprises:
(a) a heavy chain variable region (VH) comprising one, two or three CDR amino acid sequences selected form SEQ ID NOs 3, 4, 5, or 26 or variants thereof comprising one or more conservative substitutions; and/or
(b) a light chain variable region (VL) comprising one, two or three CDR amino acid sequences selected form SEQ ID NOs 6, 7, 8, or 27 or variants thereof comprising one or more conservative substitutions.

In some embodiments, the anti-Tim-3 antibody comprises:
(a) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 8 or variants thereof comprising one or more conservative substitutions;
(b) a heavy chain variable region (VH) comprising a VH-CDR1 amino acid sequence of SEQ ID NO 3 or variants thereof comprising one or more conservative substitutions, a VH-CDR2 amino acid sequence SEQ ID NO 26 or variants thereof comprising one or more conservative substitutions and a VH-CDR3 amino acid sequence of SEQ NO 5 or variants thereof comprising one or more conservative substitutions; and a light chain variable region (VL) comprising a CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 8 or variants thereof comprising one or more conservative substitutions;
(c) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions; or
(d) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 26 or variants thereof comprising one or more conservative substitutions and a VH-CDR3 amino sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions.

In some embodiments, the anti-Tim-3 antibody comprises:
(a) a heavy chain variable region (VH) comprising a VH-CDR1 amino acid sequence of SEQ ID NO 3, a VH-CDR2 amino acid sequence of SEQ ID NO 4 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 8; or
(b) a heavy chain variable region (VH) comprising a VH-CDR1 amino acid sequence of SEQ ID NO 3, a VH-CDR2 amino acid sequence of SEQ ID NO 26 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 27.

In some embodiments, the anti-Tim-3 antibody is a humanized antibody molecule.

In some embodiments, the anti-Tim-3 antibody is a humanized monoclonal antibody (mAb).

In some embodiments, the antibody comprises a heavy chain variable domain having at least 95%, 96%, 97%, 98%, 99% or 100% sequence identity with the amino acid sequence of SEQ ID NOs 9, 17, 28 or 40. In some embodiments, the antibody comprises a heavy chain variable domain having at least 95%, 96%, 97%, 98%, 99% or 100% sequence identity with the amino acid sequence of SEQ ID NOs 9, 17 or 28. In some embodiments, the anti-Tim-3 antibody comprises a light chain variable domain having at least 95%, 96%, 97%, 98%, 99% or 100% sequence identity with the amino acid sequence of SEQ ID NOs 11, 19, 30 or 36.

In some embodiments, the anti-Tim-3 antibody comprises:
(a) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 11;
(b) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 19;
(c) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 30;
(d) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 36;
(e) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 11;
(f) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 19;
(g) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 30;
(h) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 36;
(i) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 11;
(j) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 19;
(k) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 30;
(m) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 36; a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 11;
(n) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 19;
(o) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 30; or
(q) a heavy chain variable domain having at least 95%, 96%, 97%, 98%, 99% or 100% sequence identity with the amino acid sequence of SEQ ID NO 40, and a light chain variable domain having at least 95%, 96%, 97%, 98%, 99% or 100% sequence identity with the amino acid sequence of SEQ ID NO 36.

In some embodiments, the anti-Tim-3 antibody comprises:
(a) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 11;
(b) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 19;
(c) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 30;
(d) a heavy chain variable domain having at least 95%, 96%, 97%, 98%, 99% or 100% sequence identity with the amino acid sequence of SEQ ID NO 9, and a light chain variable domain having at least 95%, 96%, 97%, 98%, 99% or 100% sequence identity with the amino acid sequence of SEQ ID NO 36;
(e) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 11;
(f) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 19;
(g) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 30;
(h) a heavy chain variable domain having at least 95%, 96%, 97%, 98%, 99% or 100% sequence identity with the amino acid sequence of SEQ ID NO 17, and a light chain variable domain having at least 95%, 96%, 97%, 98%, 99% or 100% sequence identity with the amino acid sequence of SEQ ID NO 36;
(i) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 11;
(j) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 19;
(k) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 30; or;
(l) a heavy chain variable domain having at least 95%, 96%, 97%, 98%, 99% or 100% sequence identity with the amino acid sequence of SEQ ID NO 28, and a light chain variable domain having at least 95%, 96%, 97%, 98%, 99% or 100% sequence identity with the amino acid sequence of SEQ ID NO 36.

In some embodiments, the anti-Tim-3 antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30;
(d) a heavy chain variable domain comprising the amino acid sequence of SEQ ID NO 28, and a light chain variable domain comprising the amino acid sequence of SEQ ID NO 36; or
(e) a heavy chain variable domain comprising the amino acid sequence of SEQ ID NO 40, and a light chain variable domain comprising the amino acid sequence of SEQ ID NO 36.

In some embodiments, the anti-Tim-3 antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30; or
(d) a heavy chain variable domain comprising the amino acid sequence of SEQ ID NO 28, and a light chain variable domain comprising the amino acid sequence of SEQ ID NO 36.

In some embodiments, the anti-Tim-3 antibody comprises a heavy chain having at least 95%, 96%, 97%, 98%, 99% or 100% sequence identity with the amino acid sequence of SEQ IIS NO 13, 22 or 32. In some embodiments, the anti-Tim-3 antibody comprises a light chain having at least 95%, 96%, 97%. 98%, 99% or 100% sequence identity with the amino acid sequence of SEQ ID NO 15, 24, 34 or 38.

In some embodiments, the anti-Tim-3 antibody comprises:
(a) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 15;
(b) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 24;
(c) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 34;

(d) a heavy chain having at least 95%, 96%, 97%, 98%, 99% or 100% sequence identity with the amino acid sequence of SEQ ID NO 13, and a light chain having at least 95%, 96%, 97%, 98%, 99% or 100% sequence identity with the amino acid sequence of SEQ ID NO 38;
(e) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 15;
(f) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 24;
(g) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 34;
(h) a heavy chain having at least 95%, 96%, 97%, 98%, 99% or 100% sequence identity with the amino acid sequence of SEQ ID NO 22, and a light chain having at least 95%, 96%, 97%, 98%, 99% or 100% sequence identity with the amino acid sequence of SEQ ID NO 38;
(i) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 15;
(j) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 24;
(k) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 34; or
(l) a heavy chain having at least 95%, 96%, 97%, 98%, 99% or 100% sequence identity with the amino acid sequence of SEQ ID NO 32, and a light chain having at least 95%, 96%, 97%, 98%, 99% or 100% sequence identity with the amino acid sequence of SEQ ID NO 38.

In some embodiments, the anti-Tim-3 antibody comprises:
(a) a heavy chain comprising the amino acid sequence of SEQ ID NO 13, and a light chain comprising the amino acid sequence of SEQ ID NO 15;
(b) a heavy chain comprising the amino acid sequence of SEQ ID NO 22, and a light chain comprising the amino acid sequence of SEQ ID NO 24;
(c) a heavy chain comprising the amino acid sequence of SEQ ID NO 32, and a light chain comprising the amino acid sequence of SEQ ID NO 34; or
(d) a heavy chain comprising the amino acid sequence of SEQ ID NO 32, and a light chain comprising the amino acid sequence of SEQ ID NO 38.

In some embodiments, the anti-Tim-3 antibody comprises one or more of:
(a) a light chain with an Aspartic acid to Glutamic acid mutation at position 1 of SEQ NO 24;
(b) a light chain with a Leucine to Methionine mutation at position 4 of SEQ ID NO 24;
(c) a light chain with a Valine to Isoleucine mutation at position 62 of SEQ ID NO 24;
(d) a light chain with a Aspartic acid to Glutamic acid mutation at position 74 of SEQ ID NO 24;
(e) a light chain with a Methionine to Leucine mutation at position 96 of SEQ ID NO 24;
(f) a heavy chain with a Phenylalanine to Tyrosine mutation at position 59 of SEQ ID NO 22;
(g) a heavy chain with a Proline to Valine mutation at position 60 of SEQ ID NO 22;
(h) a heavy chain with a Serine to Threonine mutation at position 77 of SEQ ID NO 22; or
(i) a heavy chain with a Cysteine to Leucine mutation at position 78 of SEQ ID NO 22.

In some embodiments, the anti-Tim-3 antibody is a Fab, F(ab′)2, Fv, or a single chain Fv (ScFv).

In some embodiments, the anti-Tim-3 antibody comprises a heavy chain constant region of the subclass of IgG1, IgG4 or a variant thereof, and a light chain constant region of the type of kappa or lambda or a variant thereof.

In some embodiments, the anti-Tim-3 antibody comprises a variant heavy chain constant region of the subclass of IgG1, IgG2, IgG3, or IgG4, wherein the variant heavy chain constant region provides a reduced or eliminated effector function such as antibody-dependent cell-mediated cytotoxicity (ADCC) or complement-dependent cytotoxicity (CDC).

In some embodiments, the anti-Tim-3 antibody comprises a heavy chain constant region of human IgG1 or a variant thereof. In more preferred embodiments, the anti-Tim-3 antibody, comprises a variant heavy chain constant region of human IgG1 comprising one or more mutations selected from a group consisting of E₂₃₃P, L₂₃₄A, L₂₃₅A, L₂₃₆A and P₃₂₉A. In some embodiments, the anti-Tim-3 antibody comprises a variant human IgG1 heavy chain constant region comprising the amino acid sequence of SEQ ID NO 21, and a human kappa light chain constant region.

In some embodiments, the anti-Tim-3 antibody is isolated or recombinant.

In some embodiments, the anti-Tim-3 antibody comprises at least one antigen-binding site, or at least a variable region. In some embodiments, the anti-Tim-3 antibody comprises an antigen-binding fragment from an antibody described herein.

The term “antibody” herein is used in the broadest sense and specifically covers antibodies (including full length monoclonal antibodies) and antibody fragments so long as they recognize antigen, e.g., Tim-3, PD-1. An antibody is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies, and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ansubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs)”, which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabul; et al., National Institutes of Health, Bethesda, Md; 5<m>ed; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987). J Mol. Biol. 196:901-917 or Chothia, et al, (1989) Nature 342:878-883.

The term “hypervariable region” means the amino acid residues of an antibody that are responsible for antigen-binding. The hypervariable region comprises amino acid residues from a “CDR” (i.e., VL-CDR1, VL-CDR2 and VL-CDR3 in the light chain variable domain and VH-CDR1, VH-CDR2 and VH-CDR3 in the heavy chain variable domain). See, Kabul et al. (1991) Sequences of Proteins of Immunological Interest, 5th Ed. Public Health Service, National Institutes of Health, Bethesda, Md. (defining the CDR regions of an antibody by sequence); see also Chothia and Lesk: (1987) J. Mol. Biol. 196: 901-917 (defining the CDR regions of an antibody by structure). The term “framework” or “FR” residues means those variable domain residues other than the hypervari able region residues defined herein as CDR residues.

Unless otherwise indicated, “antibody fragment” or “antigen-binding fragment” means antigen binding fragments of antibodies, i.e., antibody fragments that retain the ability to bind specifically to the antigen bound by the full-length antibody, e.g. fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered “specific” for its intended target if its binding is determinative of the presence of the target protein in a. sample, e.g., without producing undesired results such as false positives. Antibodies or binding fragments thereof, useful in the present invention will bind to the target protein with an affinity that is at least two-fold greater, preferably at least 10-times greater, more preferably at least 20-times greater, and most preferably at least 100-times greater than the affinity with non-target proteins, An antibody herein is said to bind specifically to a polypeptide comprising a given amino acid sequence, e.g., the amino acid sequence of a mature human Tim-3 molecule, if it binds to polypeptides comprising that sequence but does not bind to proteins lacking that sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu”, “flu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The terms “cancer” or “tumor” herein mean or describe the physiological condition in mammals that is typically characterized by unregulated cell growth. Examples of cancer include but are not limited to, a lung cancer (including small-cell lung cancer, or non-small cell lung cancer), an adrenal cancer, a liver cancer, a stomach cancer, a cervical cancer, a melanoma, a renal cancer, a breast cancer, a colorectal cancer, a leukemia, a bladder cancer, a bone cancer, a brain cancer, an endometrial cancer, a head and neck cancer, a lymphoma, an ovarian cancer, a skin cancer, a thyroid tumor, or a metastatic lesion of the cancer.

The term “CDRS” means complementarity determining region(s) in an immunoglobulin variable region, defined using the Kabat numbering system, unless otherwise indicated.

### Pharmaceutical Compositions and Kits

In some aspects, this disclosure provides compositions, e.g., pharmaceutically acceptable compositions, which include an anti-Tim-3 antibody described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions herein may be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection, In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The “subject” is a mammal, e.g,, a primate, preferably a higher primate, e.g., a human (e.g., a patient having, or at risk of having, a disorder described herein).

### EXAMPLE

### Example 1. Generation of Anti-Tim-3 Monoclonal Antibodies

A number of murine anti-Tim-3 monoclonal antibodies (mAbs) were generated based on conventional hybridoma technology (de StGroth and Sheidegger, 1980, J Immunol. Methods 35:1; Mechetner, 2007, Methods Mol Biol 378:1). The mAbs with high binding activity in enzyme-linked immunosorbent assay (ELISA) and fluorescence-activated cell sorting (FACS) assay were selected for further characterization.

Tim-3 Recombinant Proteins for Immunization and Binding Assays

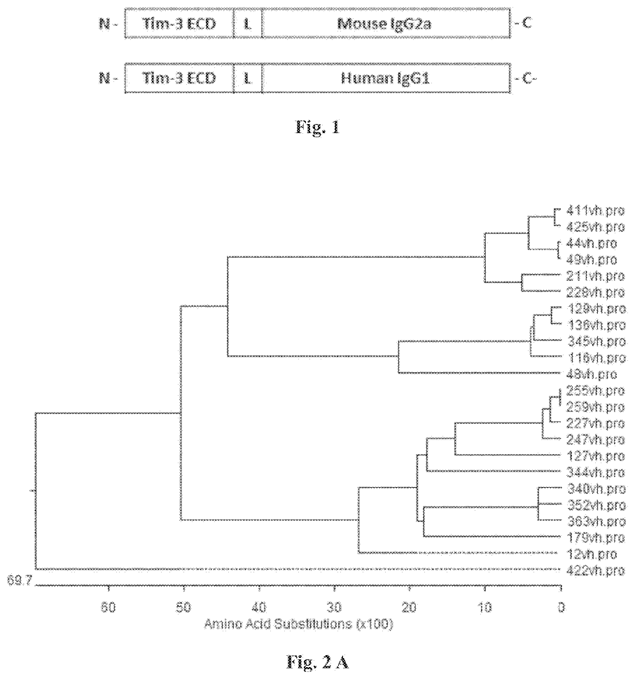

The cDNA coding for the full-length human Tim-3 (SEQ ID NO 1) was synthesized based on the GenBank sequence (Accession No: AF450242.1). The coding region of extracellular domain (ECD) consisting of amino acid (AA) 1-202 of Tim-3 (SEQ ID NO. 2) was PCR-amplifled, and cloned into a pcDNA3.1-based expression vector (Invitrogen, Carlsbad, Calif., USA) with C-terminus fused either to the Fc region of mouse IgG2a (GenBank Accession No: CAC20702) or to the Fe region of human IgG1 heavy chain (UniProtKB/Swiss-Prot Accession No: P01857), which resulted in two recombinant fusion protein expression plasmids, Tim-3-migG2a and Tim-3-huIgG1, respectively. The schematic presentation of Tim-3 fusion proteins is shown in FIG. 1. For the recombinant fusion protein production, Tim-3-mIgG2a and Tim-3-huIgG1 plasmids were transiently transfected into a HEK293-based mammalian cell expression system (developed in house) and cultured for 5-7 days in a CO₂ incubator equipped with rotating shaker. The supernatant containing the recombinant protein was collected and cleared by centrifitgation. Tim-3-mIgG2a and Tim-3-huIgG1 were purified using a Protein Ci Sepharose Fast Flow column (Cat. No.: 17061805, GE Life Sciences). Both Tim-3-mIgG2a and Tim-3-huIgG1 proteins were dialyzed against phosphate buffered saline (PBS) and stored in −80° C. freezer in small aliquots.

Stable Expression Cell Lines

To establish stable cell lines that express full-length human Tim-3 (huTim-3) or monkey Tim-3 (mkTim-3, the gene is available from ZYAGE, Cat. No.: KD-702), Tim-3 genes were cloned into a retroviral vector pFB-Neo (Cat. No.: 217561, Agilent, USA). Dual-tropic retroviral vectors were generated according to a previous protocol (Zhang T., et al., 2005, Blood 106:1544-51). Vectors containing huTim-3 and mkTim-3 were transduced into HuT78 and NK92MI cells (ATCC, Manassas, Va., USA), respectively, to generate the cell lines, HuT78/huTim-3 and NK92MI/mkTim-3. The high expression cell lines were selected by culture in complete RPMI1640 medium containing 10% FBS with G418 and FACS binding assay.

Immunization, Hybridoma Fusion and Cloning

Eight to twelve week-old Balb/c mice (HFK BIOSCIENCE CO., LTD, Beijing, China) were immunized intraperitoneally (i.p.) with 100 μl of antigen solution containing 10 μg of Tim-3-mIgG2a and a water-soluble adjuvant (Cat. No.: KX0210041, KangBiQuan, Beijing, China), The procedure was repeated three weeks later. Two weeks after the 2^{nd} immunization, mouse sera were evaluated for Tim-3 binding by ELISA and FACS, Ten days after serum screening, the mice with highest anti-Tim-3 antibody serum titers were boosted i.p. with 50 μg of Tim-3-mIgG2a. Three days after boosting, the splenocytes were isolated and fused to the murine myeloma cell line, SP2/0 cells (ATCC), using the standard techniques (Colligan E, et al., CURRENT PROTOCOLS IN IMMUNOLOGY, 1993).

Assessment of Tim-3 Binding Activity of Antibodies by ELISA and PACS

The supernatants of hybridoma clones were initially screened by ELISA as described in Methods in Molecular Biology (2007) 378:33-52 with some modifications. Briefly, Tim-3-huIgG1 protein was coated in 96-well plates. The HRP-linked anti-mouse IgG antibody (Cat. No.: 7076S, Cell Signaling Technology, USA) and substrate (Cat. No.: 00-4201-56, eBioscience, USA) were used for development, and absorbance signal at the wavelength of 450 nm was measured using a plate reader (SpectraMax Paradigm, Molecular Devices, USA). The ELISA-positive clones were further verified by FACS using either HuT78/huTim-3 or NK92mi/mkTim-3 cells described above. Tim-3-expressing cells (10⁵ cells/well) were incubated with ELISA-positive hybridoma supernatants, followed by binding with Alexa Fluro-647-labeled goat anti-mouse IgG antibody (Cat. No.: A0473, Beyotime Biotechnology, China). Cell fluorescence was quantified using a flow cytometer (Guava easyCyte 8HT, Merck-Millipore, USA).

The conditioned media from the hybridomas that showed positive signals in both ELISA and FACS screening were subjected to functional assays to identify antibodies with good functional activity in human immune cell-based assays (see following sections). The antibodies with desired functional activities were further sub-cloned and characterized.

### Subclotting and Adaptation of Hybridomas to Serum Free or Low Serum Medium

After screening primarily by ELISA, FACS and functional assays (described in Examples 7 and 8), the positive hybridoma clones were sub-cloned by limiting dilution. The top antibody subclones verified through functional assays were adapted for growth in the CDM4MAb medium (Cat. No.: SH30801.02, Hyclone, USA) with 3% FBS.

Expression and Purification of Monoclonal Antibodies

Hybridoma cells were cultured in CDM4MAb medium (Cat. No.: SH30801.02, Hyclone), and incubated in a CO₂ incubator for 5 to 7 days at 37° C. The conditioned medium was collected through centrifugation and filtrated by passing a 0.22 82 m membrane before purification. Murine antibody-containing supernatants were applied and bound to a Protein A column (Cat. No.: 17127901, GE Life Sciences) following the manufacturer's guide. The procedure usually yielded antibodies at purity above 90%. The Protein A-affinity purified antibodies were either dialyzed against PBS or further purified using a HiLoad 16/60 Superdex200 column (Cat. No.: 17531801, GE Life Sciences) to remove aggregates. Protein concentrations were determined by measuring absorbance at 280 nm. The final antibody preparations were stored in aliquots in −80° C. freezer.

### Example 2. Cloning and Sequence Analysis of Tim-3 Antibodies

Murine hybridoma cells were harvested to prepare total RNAs using Ultrapure RNA kit (Cat. No.: 74104, QIAGEN, Germany) based on the manufacturer's protocol. The 1^{st} strand cDNAs were synthesized using a cDNA synthesis kit from Invitrogen (Cat. No.: 18080-051) and PCR amplification of Vh and Vk genes of murine mAbs was performed using a PCR kit (Cat. No.: CW0686, CWBio, Beijing, China). The oligo primers used for antibody cDNAs cloning of heavy chain variable region (Vh) and kappa light chain variable region (Vk) were synthesized based on the sequences reported previously (Brooks et al., 2001 Mol Med 7:461). PCR products were then subcloned into the pEASY-Blunt cloning vector (Cat. No.: CB101-02, TransGen, China) and sequenced. The amino acid sequences of Vh and Vk regions were deduced from the DNA sequencing results.

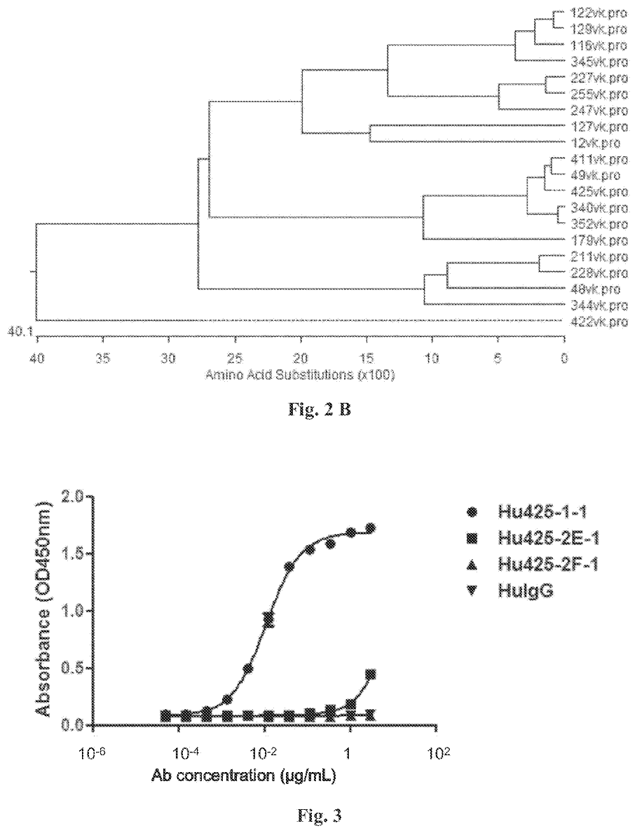

The murine mAbs were analyzed by comparing sequence homology and grouped based on sequence similarity (FIG. 2). Complementary determinant regions (CDRs) were defined based on the Kabat (Wu and Kabat, 1970, J. Exp. Med. 132:211-250) and IMGT (Lefranc, 1999, Nucleic Acids Research 27:209-212) system by sequence annotation and by internet-based sequence analysis (http://www.imgt.org/IMGT_vquest/share/textes/index.html). The amino acid sequences of a representative top clone mu425 (Vh and Vk) were listed in Table 1 (SEQ NOs. 9 and 11). The CDR sequences of mu425 were listed in Table 2 (SEQ ID NOs 3-8).

TABLE 1
Amino acid sequences of mu425 VH and VK regions

| | | SEQ ID |
|---|---|---|
| | Sequence | NO |
| mu425 VH | EVKLVESGGGLVKPGGSLKLSCAASGFTFSRYAMSWVRQIPEKRLEW | 9 |
| | VAAISSGGSLYFPDSVKGRFTISRDNARNICYLQMNSLRSDDTAMYYC | |
| | ARGREADGGYFDYWGQGTTLTVSS | |
| mu425 VK | DIVLTQSPASLAVSLGQRATISCRASESVEYYGTSLMQWYQQKPGQPP | 11 |
| | KLLIYAASNVESGVPARFSGSGSGTDFSLNIHPVEEDDIAMYFCQQSM | |
| | KVPLTFGAGTKLELK | |

TABLE 2
CDR sequences (amino acids) of mu425 VH and VK regions

| | | SEQ ID | | SEQ ID | | SEQ ID |
|---|---|---|---|---|---|---|
| | CDR1 | NO | CDR2 | NO | CDR3 | NO |
| mu425, VH | RYAMS | 3 | AISSGGSLYFPDSVKG | 4 | GREADGGYFDY | 5 |
| mu425, VK | RASESVEYYGTSLMQ | 6 | AASNVES | 7 | QQSMKVPLT | 8 |

### Example 3. Affinity Determination of Purified Murine Anti-Tim-3 Antibodies by SPR

The Tim-3 antibodies with high binding activities in ELISA and FACS, as well as with potent functional activities in the cell-based assays (described in Examples 7 and 8) were characterized for their binding kinetics by SPR assays using BIAcore™ T-200 (GE Life Sciences). Briefly, anti-human IgG antibody was immobilized on an activated CM5 biosensor chip (Cat. No.: BR100530, GE Life Sciences). Human Fc-tagged Tim-3 IgV domain was flowed over the chip surface and captured by anti-human IgG antibody. Then a serial dilution (0.36 nM to 90 nM) of purified murine antibodies were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of top mAbs including mu425, mu44, mu 225 and mu411, were shown in Table 3.

TABLE 3
Comparison of hybridoma antibody
binding affinities by SPR

| Antibodies | kₒₙ (M⁻¹ s⁻¹) | k_{off} (s⁻¹) | K_{D} (nM) |
|---|---|---|---|
| mu425 | 1.59 × 10⁶ | 9.33 × 10⁻⁵ | 0.058 |
| mu44 | 1.24 × 10⁶ | 1.86 × 10⁻⁴ | 0.150 |
| mu255 | 5.52 × 10⁵ | 7.84 × 10⁻⁴ | 1.42 |
| mu411 | 1.44 × 10⁶ | 3.36 × 10⁻⁴ | 0.234 |

### Example 4. Humanization of the Murine Anti-Human Tim-3 mAb mu425

mAb Humanization and Engineering

For humanization of mu425, human germline IgG genes were searched for sequences that share high degrees of homology with the cDNA sequences of mu425 variable regions by blasting the human immunoglobulin gene database in IMGT (http://www.imgt.org/IMGT_vquest/share/textes/index.html) and NCBI (http://www.ncbi.nlm.nih.gov/igblast/) websites. The human IGVH and IGVK genes that are present in human antibody repertoires with high frequencies (Glanville, 2009, PNAS 106:20216-20221) and are highly homologous to mu425 were selected as the templates for humanization. Before humanization, mu425 heavy and light chain variable domains were fused to a modified human IgG1 constant region termed as human IgG1mf (SEQ ID NO. 21) and a human kappa constant region, respectively. IgG1mf (SEQ ID NO: 21) is an IgG1 mutant containing a combination of mutations, E₂₃₃P, L₂₃₄A, L₂₃₅A, L₂₃₆Δ and P₃₂₉A (amino acid numbering is based on EU system) as compared to wild-type human IgG1.

Humanization was carried out by CDR-grafting (Methods in Molecular Biology, Vol 248: Antibody Engineering, Methods and Protocols, Humana Press) and the humanized antibodies (hu425s) were engineered in the human IgG1 format. In the initial round of humanization, mutations from murine to human amino acid residues in framework regions were guided by the simulated 3D structure, and the murine framework residues of structural importance for maintaining the canonical structures of CDRs were retained in the 1^{st} version of humanized antibody 425, hu425-1-1 (the amino acid sequences of the heavy chain and light chain are set for in SEQ ID NOs. 22 and 24). Specifically, CDRs of mu425 Vk were grafted into the frameworks of human germline variable gene IGVK3-15 with 4 murine framework residues (D1, L4, V62 and D74) retained (the amino acid sequences of the light chain variable domain is set for in SEQ ID NO. 19). CDRs of mu425 Vh were grafted into the frameworks of human germline variable gene IGVH3-7 with 1 murine framework (C₇₈) residue retained (the amino acid sequences of the heavy chain variable domain is set for in SEQ ID NO. 17)

Hu425-1-1 was constructed as human full-length antibody format using in-house developed expression vectors that contain constant regions of a human IgG1 variant termed as IgG1mf (SEQ ID NO. 21) and kappa chain, respectively, with easy adapting subcloning sites. Expression and preparation of hu425-1-1 antibody was achieved by co-transfection of the above two constructs into 293G cells and by purification using a protein A column (Cat. No.: 17543802, GE Life Sciences). The purified antibodies were concentrated to 0.5-5 mg/mL in PBS and stored in aliquots in −80° C. freezer.

Based on hu425-1-1 template, we made a number of single-mutations converting the retained murine residues in framework regions of Vk to corresponding human germline residues, which include D1E, L4M, V62I and D74E in Vk. The resulted hu425-1-2a (D1E), hu425-1 -2b (L4M), hu425-1-2c (V62I) and hu425-1-2d (D74E) all had similar binding and functional activities to hu425-1-1. In order to further improve humanization level of the heavy chain, we also changed the retained residue C78 and the C-terminal part of H-CDR2 (Kabat's definition) from murine sequence to corresponding human germline residues. Specifications of the 3 humanized antibodies were hu425-2A-1 (F59Y in Vh), hu425-2B-1 (P60V in Vh) and hu425-2C-1 (C78L in Vh). All humanization mutations were made using primers containing mutations at specific positions and a site directed mutagenesis kit (Cat. No. FM111-02, TransGen, Beijing, China). The desired mutations were verified by sequence analysis. These hu425 valiant antibodies were tested in binding and functional assays as described previously. Comparing to hu425-1-1, hu425-2B-1 had significantly reduced binding affinities and functionalities (data not shown) while the rest versions of hu425 humanized variants had similar binding and functional activities to hu425-1-1.

Hu425 antibodies were further engineered by introducing mutations in CDRs and framework regions to improve molecular biochemical and biophysical properties for therapeutic use in human. The considerations include amino acid compositions, heat stability (Tₘ), surface hydrophobicity and isoelectronic points (pIs) while maintaining functional activities.

Taken together, two well-engineered versions of humanized monoclonal antibodies, hu425-2-2 (the amino acid sequences of the heavy chain and light chain variable domains are set for in SEQ ID NOs. 28 and 30) and hu425-2-3b (the amino acid sequences of the heavy chain and light chain variable domains are set for in SEQ ID NOs. 28 and 36), were derived from the mutation process described above, and characterized in details. Both hu425-2-2 and hu425-2-3b comprise a mutation in H-CDR2 (SEQ ID NO: 26) and in L-CDR3 (SEQ ID NO: 27). The amino acid sequences of heavy/light chain variable regions and six CDRs of hu425-2-2 and hu425-2-3b are listed in Table 4 and Table 5 below. The results showed that both hu425-2-3b and hu425-2-2 were very similar in binding affinity and functional activities such as inhibiting the Tim-3 mediated downstream signaling.

TABLE 4
Amino acid sequences of VH and VK regions of hu425-2-2 and
hu425-2-3b

| | Sequence | SEQ ID NO |
|---|---|---|
| hu425-2-2 | EVQLVESGGGLVQPGGSLRLSCAASGFTFSRYAMSWVRQAP | 28 |
| VH | GKGLEWVAAISSGGSLYYPDSVKGRFTISRDNAKNTLYLQM | |
| | NSLRAEDTAVYYCARGREADGGYFDYWGQGTLVTVSS | |
| hu425-2-2 | EIVMTQSPATLSVSPGERATLSCRASESVEYYGTSLMQWYQ | 30 |
| VK | QKPGQAPRLLIYAASNVESGIPARFSGSGSGTEFTLTISSLQSE | |
| | DFAVYYCQQSLKVPLTFGGGTKVEIK | |
| hu425-2-3b | EVQLVESGGGLVQPGGSLRLSCAASGFTFSRYAMSWVRQAP | 28 |
| VH | GKGLEWVAAISSGGSLYYPDSVKGRFTISRDNAKNTLYLQM | |
| | NSLRAEDTAVYYCARGREADGGYFDYWGQGTLVTVSS | |
| hu425-2-3b | EIVLTQSPATLSVSPGERATLSCRASESVEYYGTSLMQWYQQ | 36 |
| VK | KPGQAPRLLIYAASNVESGIPARFSGSGSGTEFTLTISSLQSED | |
| | FAVYYCQQSLKVPLTFGGGTKVEIK | |

TABLE 5
CDR sequences (amino acids) of VH and VK regions of hu425-2-2 and
hu425-2-3b

| | | SEQ ID | | SEQ ID | | SEQ ID |
|---|---|---|---|---|---|---|
| | CDR1 | NO | CDR2 | NO | CDR3 | NO |
| hu425-2-2 VH | RYAMS | 3 | AISSGGSLYYPDSVKG | 26 | GREADGGYFDY | 5 |
| hu425-2-2 VK | RASESVEYYGTSLMQ | 6 | AASNVES | 7 | QQSLKVPLT | 27 |
| hu425-2-3b VH | RYAMS | 3 | AISSGGSLYYPDSVKG | 26 | GREADGGYFDY | 5 |
| hu425-2-3b VK | RASESVEYYGTSLMQ | 6 | AASNVES | 7 | QQSLKVPLT | 27 |

For affinity determination, Fabs derived from the series of hu425 mAbs were prepared using Pierce Fab Preparation Kit (Cat. No. 44985, ThermoFisher Scientific), and used in the affinity—assay based on surface plasmon resonance (SPR) technology. The results of SPR-determined binding profiles of anti-Tim-3 Fab antibodies were summarized in Table 6. The Fabs of hu425-2-2 and hu425-2-3b have very similar binding profiles with average dissociation constant at 0.419 nM and 0.361 nM, respectively, which are close to that of hu425-1-1.

TABLE 6
Comparison of hu425 Fab binding affinities by SPR

| Anti-Tim-3 | Test 1 | | | Test 2 | | | Mean |
|---|---|---|---|---|---|---|---|
| Fabs | kₒₙ (M⁻¹s⁻¹) | k_{off} (s⁻¹) | K_{D} (nM) | kₒₙ (M⁻¹s⁻¹) | k_{off} (s⁻¹) | K_{D} (nM) | K_{D}(nM) |
| ch425* | 1.67 × 10⁶ | 2.04 × 10⁻⁴ | 0.122 | | | | NA** |
| hu425-1-1 | 6.79 × 10⁵ | 1.08 × 10⁻⁴ | 0.159 | 1.26 × 10⁶ | 4.35 × 10⁻⁴ | 0.346 | 0.253 |
| hu425-2-2 | 1.71 × 10⁶ | 7.40 × 10⁻⁴ | 0.434 | 1.97 × 10⁶ | 7.95 × 10⁻⁴ | 0.404 | 0.419 |
| hu425-2-3b | 1.60 × 10⁶ | 5.70 × 10⁻⁴ | 0.356 | 1.75 × 10⁶ | 6.40 × 10⁻⁴ | 0.366 | 0.361 |

*ch425 is comprised of mu425 variable domains fused to human IgG1 mf/mouse kappa constant regions (the amino acid sequences of the heavy chain and light chain are set for in SEQ ID NOs. 13 and 15)
**NA: not available.

All the humanized antibodies shown above were also confirmed for functional activities on primary human immune cells isolated from healthy donors (described in Example 8).

### Example 5 Affinity Comparison of Anti-Tim-3 Antibodies by SPR

Hu425-2-3b and two known Titn-3 antibodies, Ab1 (comprising a heavy chain variable region of SEQ ID NO: 40 and a light chain variable region of SEQ ID NO: 41) and Ab2 (comprising a heavy chain variable region of SEQ ID NO: 42 and a light chain variable region of SEQ ID NO: 43), were generated in human IgG4 format with S228P mutation and characterized for their binding kinetics by SPR assays using BIAcore™ T-200 (GE Life Sciences).

Briefly, anti-human Fab antibody was immobilized on an activated CM5 biosensor chip (Cat.: BR100530, GE Life Sciences). Anti-Tim-3s were flown through the chip surface and captured by anti-human Fab antibody. Then a serial dilution (0.12 nM to 30 nM) of Tim-3-his were flown over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. Hu425-2-3b, Ab1 and Ab2 displayed comparable binding affintiy. The antibodies, specifically hu425-2-3b binds to Tim-3 in a dose-dependent manner with a nanomolar KD.

TABLE 7
Comparison of binding affinities
of anti-Tim-3s by SPR

| Anti-Tim-3 | Ka (1/Ms) | Kd (1/s) | KD (M) |
|---|---|---|---|
| Hu425-2-3b | 5.28E+05 | 7.78E−04 | 1.47E−09 |
| Ab1 | 4.44E+05 | 1.20E−04 | 2.71E−10 |
| Ab2 | 6.27E+05 | 0.0122 | 1.94E−08 |

### Example 6. The Contribution of mu425/hu425 CDRs to Tim-3 Binding

During the process of humanization, several variants with single amino acid mutation from hu425-1-1 were generated. Two of such mutant variants almost completely abolished binding to Tim-3 as seen in FIG. 3, yet each only has a neutral (chemically similar) amino acid substitution, D102F, (the mAb was termed hu425-2E-1) and G103A (termed hu425-2F-1), in the CDR3 of heavy chain. Those findings supported the notion that the CDR3 of heavy chain in the mu425 humanized antibodies has significant contribution to the Tim-3-binding functionality.

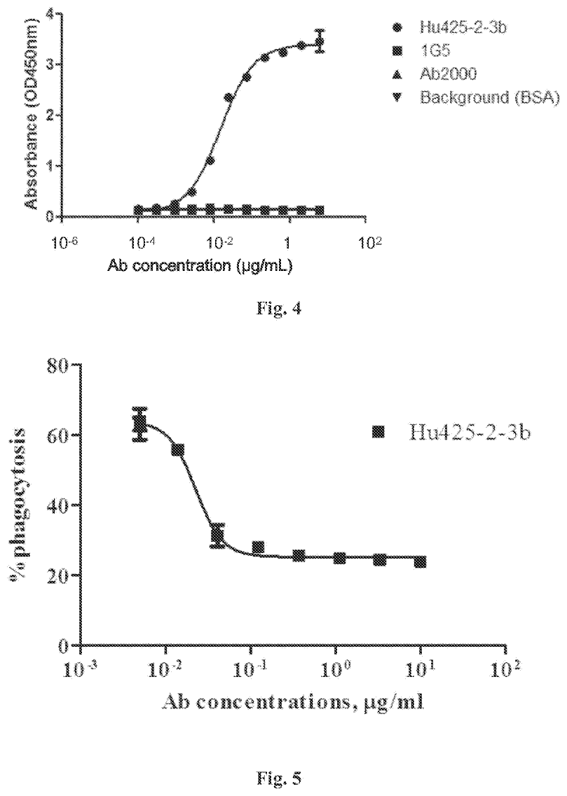

On the other hand, the CDR1 and CDR2 of mu425/hu425 light chain were identical to the mouse germline gene IGKV3-1. Several murine antibodies were also found containing the same murine germline CDR1 and CDR2 sequences in their light chain variable regions, e.g. Ab2000 (U.S. Pat. No. 7,989,597) and mAb 1G5 (U.S. Pat. No. 7,563,874). We investigated whether these antibodies could also bind to Tim-3. For this purpose, Ab2000 and 1G5 were generated with human IgG1mf format and evaluated for Tim-3-mIg binding by ELISA. As shown in FIG. 4, neither Ab2000 nor 1G5 was capable of producing any Tim-3 binding signal in contrast to hu425-2-3b. Therefore, the CDR1 and CDR2 of murine germline gene IGKV3-1 are not sufficient for Tim-3 binding.

To further evaluate the contribution of CDRs to Tim-3 binding, two hybrid antibodies were created by exchanging the heavy chain and light chain of the antibody hu425-2-3b and the antibody Ab1 with each other. Both hu425-2-3b and Ab1 are mouse derived anti-Tim-3s, and they share identical L-CDR1 and L-CDR2 to those of mouse germline IGKV3-1. The heavy chain of hu425-2-3b and the light chain of Ab1 were co-expressed to generate a hybrid antibody 425 HC/Ab1 LC, while hybrid antibody Ab1 HC/425 LC was prepared by co-expression of heavy chain of Ab1 and light chain of hu425-2-3b. The binding activities of hybrid antibodies were analyzed by BioLayer Interferometry (ForteBio Octet). The hybrid antibodies were captured by protein A tips and then dipped in Tim-3-his solution for BLI binding signal analysis. It was observed that Ab1 HC/425 LC retained Tim-3 binding capability, while captured 425 HC/Ab1 LC failed to produce significant binding signal. It suggested that the LC-CDR3 of hu425-2-3b is required for its binding to Tim3 while its L-CDR1 and L-CDR2 is not sufficient for Tim3-binding, or may not be required for binding to Tim3.

### Example 7. Blockade of Tim-3-Mediated Phagocytosis by Anti-Tim-3 Antibodies

Tim-3 has been shown to bind to phosphatidylserine (PtdSer) via its IgV domain and mediate phagocytosis of apoptotic cells (DeKruyff et al., J. Immunol, 2010, 184:1918-1930; Nakayama et al., Blood, 2009, 113: 3821-3830), PtdSer is a phospholipid that is confined to the inner leaflet of the plasma membrane in normal mammalian cells but becomes exposed on the outer surface of apoptotic cells, PtdSer is involved in the immunosuppression in tumor microenvironment by preventing immune responses (Fadok et al., J. Clin Invest, 1998, 101:890-898; Frey et al., Semin Immunopathol., 2011, 33:497-516). In vivo administration of anti-Tim-3 antibodies leads to less clearance of apoptotic cells, increased local inflammation and breaking of immune tolerance, suggesting that PtdSer-Tim-3 axis is involved in immune suppression in vivo (Chabtini et al., J. Immunol 190:88-96, 2013; Nakayamo et al., Blood 113: 3821-30, 2009).

To determine whether anti-Tim-3 antibodies could block Tim-3-mediated phagocytosis, a cell-based assay was established using the sensor and functional readout cell line, THP-1/Tim-3, i.e., THP-1 (ATCC, a human monocyte cell line) stably transfected with the full-length Tim-3 gene. In this assay, HuT78 (ATCC) were induced to undergo limited apoptosis by overnight treatment with 2% ethanol, followed by labeling with USE dye (Invitrogen, 1 μM) according to the manufacturer's instruction. THP-1/Tim-3 cells were then co-cultured with the CFSE-labeled apoptotic HuT78 cells for 6 hours in the presence of anti-Tim-3 humanized antibodies. The Tim-3-mediated phagocytosis was determined as percentage of CFSE THP-1/Tim-3 to total THP-1/Tim-3 cells (gated on CD3⁻ population). As shown in FIG. 5, hu425-2-3b dose-dependently inhibited phagocytosis of apoptotic HuT78 cell by THP-1/Tim-3 cells.

### Example 8. Activation of IFN-γ Secretion by Anti-Tim-3 Antibodies in Primary Human PBMCs Co-Cultured with T-Cell Engager-Expressing Tumor Cells

It was reported that both Tim-3 and PD-1 are inhibitory receptors expressed in activated T cells, which might function to induce T-cell exhaustion (Anderson A. C. et al., 2016, Immunity 44:989-1004). Tim-³⁺ CD4 and CD8 T-cells from cancer patients indeed secrete much less Th1 cytokine, IFN-γ, than Tim-3⁻ T cells (Arai et al., 2012, Yonago Acta medica 55:1-9; Xu B, et al., 2015, Oncotarget 6:20592-603).

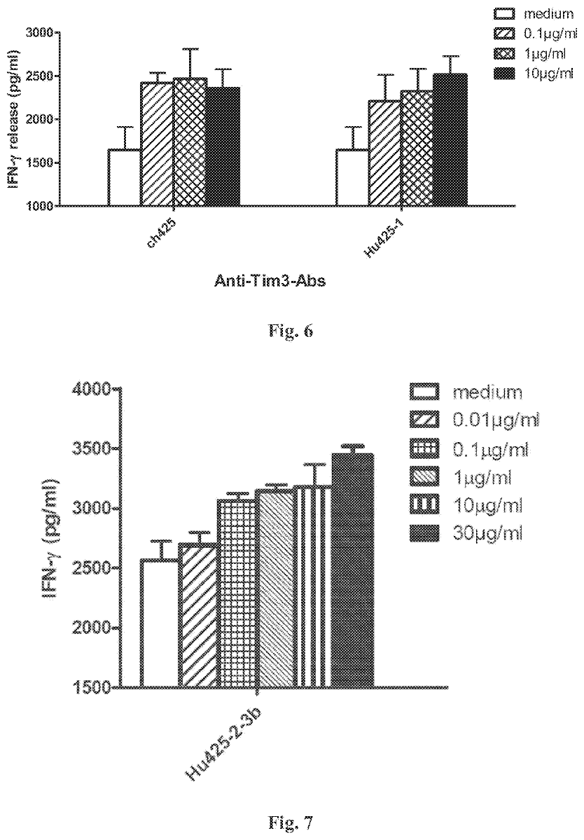

We have explored the function of Tim-3 and anti-Tim-3 antibodies using anti-CD3 mAb OKT3-actived T-cells in human PBMCs. Peripheral blood mononuclear cells (PBMCs) were isolated from healthy donors using density gradient centrifugation protocol with Histopaque-1077 (Cat. No.: 10771, Sigma). Three days prior to the assay, PBMCs were stimulated with 40 ng/mL of anti-CD3 triAb OKT3 (Cat. No.: 16-0037. eBioscience, USA) to amplify the CD3⁺ T-cells, which are used as effector cells. The target cell termed A549/OS8 was a lung cancer cell line A549 (ATCC) stably-transfected with a T-cell engager (OS8) based on the method described in U.S. Pat. No. 8,735,553. OS8 contains a sav of anti-human CD3 mAb OKT3 at the N-terminal domains, which directly interact with TCR/CD3 complex and activate T cells. The effector cells, PBMCs, were co-cultured with the mitotnycin-C-briefly treated A549/OS8 target cells to mimic the response of activated T cells to tumor cells upon engagement of TCR/CD3 complex. The assay was performed in the presence or absence of anti-Tim-3 antibodies in 96-well flat-bottom plates. After 15-18 hours of co-culture, culture supernatants were assayed for IFN-γ level by ELISA using Ready-Set-Go! ELISA kits (Cat. No.: 88-7316, eBiosciences). As shown in FIG. 6, when the A549/OS8 target cells were co-cultured with the effector cells, anti-Tim-3 antibodies (ch425 and hu425-1-1) induced increased IFN-γ secretion in the effector cells.

### Example 9. Activation of CMV-Specific Human T Cells by Anti-Tim-3 Antibodies

The functional activity of the Tim-3 antibodies were further assessed using naturally derived T-cells that recognize human CMV PP65 peptide (NLVPMVATV, 495-503, HLA-A2.1-restricted) (Boeckli M, Rocca M and Geballe A P, 2011, J Clin Invest. 121:1673-80). Briefly, PBMCs from healthy donors were initially screened by FACS using anti-HLA-A2 tnAb. HLA-A2⁺ PRMCs were then simulated with PP65 peptide (>98% purity, synthesized by GL Biochem, Shanghai) in the complete RPMI with 10% FBS for a week.

Target cell line A549/A2.1 was established by stable transfection of HLA-A2.1. After 30 minutes of mitomycin-c (100 μg/ml) treatment and pp65 peptide (5 μg/ml) pulse, A549/A2.1 cells (10⁴) were co-cultured with an equal number of pp65-sensitized PRNICs in 96-well plates overnight in the presence or absence of anti-Tim-3 antibodies or controls. IFN-γ in the culture supernatant was determined by ELISA. All conditions were performed in triplicates. As shown in FIG. 7, hu425-2-3b promotes pp65-specific T cells to secrete IFN-γ into the cell culture supernatant.

### Example 10. Anti-Tim-3 Antibodies Enhanced NK Cell-Mediated Cytotoxicity

Tim-3 is known to be constitutively expressed on natural killer (NK) cells at relatively high levels (Ndhlovu L C, et al., 2012, Blood 119:3734-43; da Silva I P, et al., 2014, Cancer Innnunol Res. 2:410-22). In melanoma, higher Tim-3 expression on NK cells was found to be associated with advanced tumor stages and poor prognosis. In addition, the NK cell function seemed to be influenced by Tim-S activity (daSilva I P, et al., 2014, Cancer Immunol Res. 2:410-22).

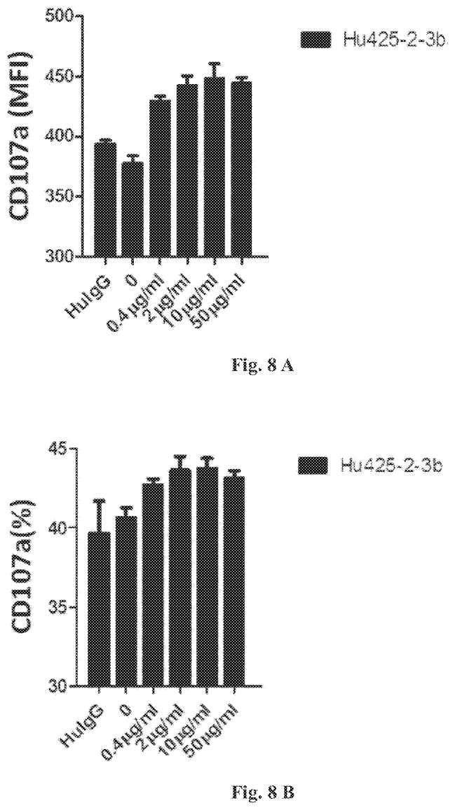

To confirm whether humanized anti-Tim-3 antibodies could promote NK-medi ated cytotoxicity, primary NK cells were isolated from PBMCs of healthy donors using an NK cell isolation kit from Biotec (Germany) according to manufacturer's instruction. After one day stimulation with human IL-2. (1000 U/ml), NK cells were co-cultured with K562 cells in the presence of anti-Tim-3 antibodies, brefeldin A and anti-CD107a-APC (eBioscience) at 37° C. for 5 hr. CD107a expression on CD3⁻CD56⁺ NK cells was quantified by flow cytometry. The results showed that anti-Tim-3 antibody hu425-2-3b increased CD107a expression measured by mean fluorescence intensity (MFI) and percentage of cell numbers (FIG. 8).

### Example 11. Anti-Tim-3 Antibodies Reduce the Surface Expression of Tim-3 Receptor

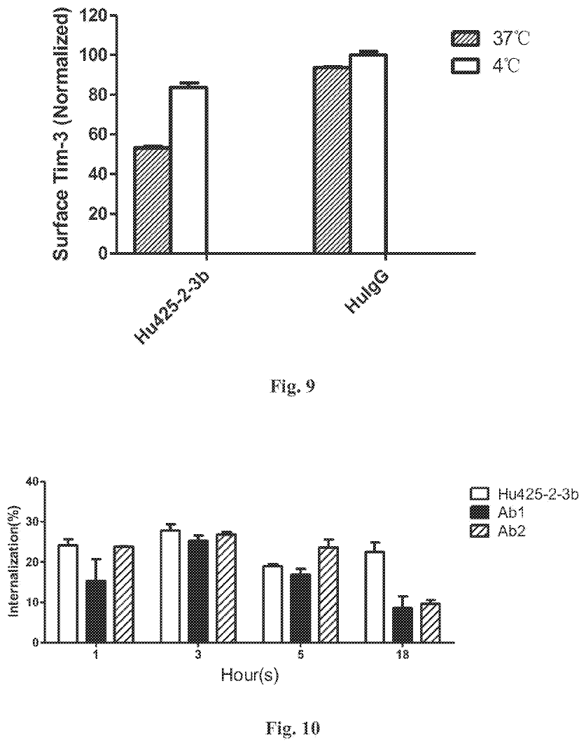

To address the possibility of hu425-2-3b-including Tim-3 internalization, NK cells from healthy donors were first incubated with hu425-2-3b (10 μg/mL) in complete RPMI1640 media at either 37° C. or 4° C., for 1 hr. Surface expression of Tim-3 receptor was determined by staining with a non-competing Tim-3 Ab mu420 (generated in house), followed by staining with goat anti-mouse IgG-APC (Biolegend), As shown in FIG. 9, hu425-2-3b at 37° C. caused a significant reduction of Tim-3 surface expression compared to the negative control human IgG-treated cells. The reduction was likely due to the anti-Tim-3 antibody-induced receptor internalization because it showed clear temperature dependence in a short time period. The results clearly demonstrated that hu4-25-2-3b induced down-regulation of Tim-3 receptor, probably due to the internalization of Tim-3. By inducing Tim-3 internalization, hu425-2-3b is expected to reduce the interactions of Tim-3 to its multiple ligands (such as galectin-9 and PtdSer).

### Example 12. Anti-Tim-3 Antibodies Exhibits Increased Internalization Rate

To further investigate the internalization of the antibodies, anti-Tim-3 antibody-induced internalization was determined using a Tim-3-expressing NK cell line (NK92MI/huTim-3) at different time points (1, 3, 5 and 18 hr). In brief, anti-Tim-3 antibodies (10 μg/ml), including hu425-2-3b, Ab1 and Ab2, were incubated with NK92MI/huTim-3 (5×10⁴) at either 37° C. or 4° C. for 18 hr. Surface expression of Tim-3 receptor was determined by staining with goat anti-human IgG-FITC. The % of internalization was calculated as the reduction (%) of Tim-3 surface expression at 37° C. as compared with the expression level at 4° C. As shown in FIG. 10, during shorter incubation (1-5 hr), hu425-2-3b induced comparable levels of Tim-3 internalization as Ab1 and Ab2. It did demonstrate a significantly persistent internalization than both Ab1 and Ab2 after 18 hours of incubation, suggesting the persistent activity of Tim-3 internalization by hu425-2-3b.

### Example 13. Humanized Anti-Tim-3 mAbs Stimulate Immune Cells Alone and in Combination with PD-1 mAb

It has been reported that the immune inhibitory receptors PD-1 and Tim-3 were up-regulated in “dysfunctional” tumor antigen-specific CD8⁺ T cells in patients with advanced tumors and chronic viral infections (Fourcade J, et al., 2010, J. Exp Med. 207:2175-86; Thommen D S, et al., 2015, Cancer Immunol Res. 3:1344-55; Jin H T et al., 2010, Proc Natl Acad Sci USA. 107:14733-8). Simultaneous blockade of both PD-1 and Tim-3 receptors could expand vaccine induced NY-ESO-1-specific CD8⁺ T cells (Foureade J., et al., 2014. Cancer Res. 74:1045-55). A conventional T-cell response assay, mixed lymphocyte reaction (MLR), was set up to characterize the potential costimulatory effects by the anti-Tim-3 and anti-PD-1 antibodies. In brief, “stimulator PBMCs” were pre-treated with mitomycin-c (100 μg/ml, Sigma) and co-cultured with “responder PBMCs” of a different donor at one to one ratio in the complete RPMI1640 media with 10% AB serum (Sigma) plus anti-Tim-3 and/or anti-PD-1 mAb 317-4B6 (also named hu317-4B6, 317-4B6/IgG4mt10, described in U.S. Pat. No. 8,735,553). The reactions were carried out in 96-well flat-bottom plates for 4 days with triplicate data point setting for each condition. IFN-γ secretion in the cell culture supernatant was analyzed as readout.

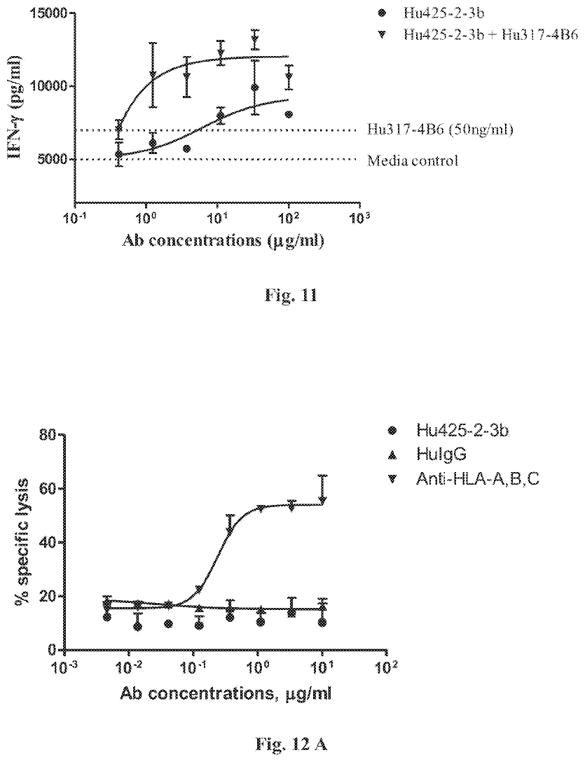

The results showed that hu425-2-3b significantly enhanced IFN-γ production in the MLR dose-dependently. Combination of hu425-2-3b with 317-4B6 (50 ng/ml) lead to a greater increase in IFN-γ production than hu425-2-3b or anti-PD-1 antibody alone, demonstrating the co-stimulating effects of the anti-Tim-3 mAb with the anti-PD-1 mAb (FIG. 11).

Mitomycin-C-pretreated “stimulator PBMCs” were co-cultured with “responder PBMCs” in the presence of anti-Tim-3 mAb, either hu425-2-3b or hu425-2-3b, plus an anti-PD-1 Ab hu317-4b6 (50 ng/ml) in 96-well flat-bottom plates for 4 days. IFN-γ in the supernatant was determined by ELISA. All conditions were performed in triplicates. Results were shown in mean+SD.

### Example 14. Hu425-2-3b did not have ADCC and CDC Effector Functions

The ability of hu425-2-3b to induce ADCC and CDC was determined using in vitro assay as described below. IgG1mf (SEQ ID NO: 21) is an IgG1 mutant containing a combination of mutations, E₂₃₃P, L₂₃₄A, L₂₃₅A, L₂₃₆A and P₃₂₉A (amino acid numbering is based on EU system). These mutations are designed to eliminate the binding of Fc to all FcγRs as well as C1q.

ADCC (Antibody-Dependent Cell-Mediated Cytotoxicity).

A classical ADCC assay was set up to determine whether hu425-2-3b could induce ADCC. The assay effector cell line, NK92MI/CD16V cells, was generated from NK92MI cells (ATCC) by co-transducing expression plasmids containing CD16_{V158} (V158 allele) and FcRγ cDNAs. Tim-3-expressing T cell line, HuT78/Tim-3, was used as target cells. The effector cells (4×10⁴) were co-cultured with an equal number of target cells, for 5 hours in the presence of hu425-2-3b or control antibodies, either the positive control anti-MEC-I A, B, C (Biolegend.) or a negative control human IgG. Cytotoxicity was determined by lactate dehydrogenase (LDH) release assay using the CytoTox 96 Non-Radioactive Cytotoxicity Assay kit (Promega, Madison, Wis.). Specific lysis was determined by the following equation.

$$\% Specific lysis = \frac{\begin{matrix}Experimental - Effector Spontaneous - \\ Target Spontaneous\end{matrix}}{Target Maximum - Target Spontaneous} \times 100$$

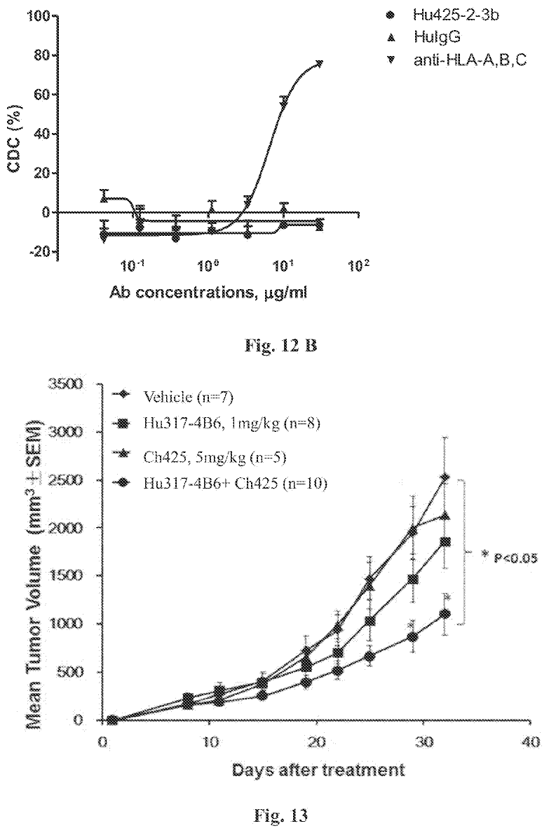

The results confirmed that hu425-2-3b had the same basal level of ADCC as that of negative control, whereas anti-MEC-I A, B, C induced ADCC in a dose-dependent manner (FIG. 12A).

### CDC (Compleinent-Dependent Cytotoxicity)

Whether hu425-2-3b would trigger CDC was determined using pre-activated human PBMCs and fresh autologous sera from healthy donors. Cell lysis by CDC was determined by a Celltiter glo assay kit (Promega, Beijing, China). In brief, PBMCs from healthy donors were pre-activated with PHA (10 μg/ml, Sigma) for 3 days, and then were incubated in RPMI1640 plus autologous serum (15%) and hu425-2-3b or control antibodies (0.04-30 μg/mL) overnight at 37° C. The cell death due to CDC was assayed by the decrease of ATP released from viable cells after cell lysis at the end of reaction. Anti-MHC-I A, B, C was used as a positive control. The fluorescence readout was conducted using a 96-well fluorometer (PHERA Star FS, BMG LABTECH), and the CDC activities were calculated from the relative fluorescence unit (RFU) readout as follows: % CDC activity=[(RFU test−RFU background)/(RFU at total cell lysis−RFU background)]×100. The experimental results demonstrated that hu425-2-3b had no detectable CDC with PBMCs isolated from two different donors. In contrast, the positive control antibody, anti-MHC-I, induced significant CDC activity (FIG. 12B).

The results showed that hu425-2-3b eliminate ADCC and CDC effector functions while maintaining optimal physicochemical properties.

### Example 15. Anti-Tim-3 Antibody in Combination with an Anti-PD-1 Antibody Inhibits Tumor Growth in a Mouse Xenograft Cancer Model

The potential anti-cancer activity of the Tim-3 antibody was evaluated in combination with anti-PD-1 antibody by a xenograft cancer model, in which immune-compromised mice were implanted with human cancer cells and allogeneic PBMCs. Briefly, NOD/SCID mice were pre-treated with cyclophosphamide (150 mg/kg) for 2 days before tumor inoculation. Human PBMCs were isolated from peripheral blood of healthy volunteers, mixed with A431 epidermoid carcinoma cells (Cat. No. CRL-1555, ATCC) in Matrigel, and injected subcutaneously into the animals. Starting from day 0, animals were randomly assigned into 4 groups with 5-10 mice per group. Mice were treated once a week (QW) via i.p. injection with vehicle (PBS), 5 mg/kg anti-Tim-3 mAb chimeric 425 (ch425), anti-PD-1 antibody 317-4B6 (1 mg/kg) or combination therapy (5 mg/kg ch425 plus 1 mg/kg of 317-4B6) for 4 weeks. Tumor size of individual mouse was recorded twice weekly, with mice being monitored daily for clinical signs of toxicity for the duration of the study. Tumor volumes were calculated using the formula: [D×(d²)]/2, in which D represents the long diameter of the tumor, and d represents the short diameter. All animal studies were performed following Beigene Animal Care and Use Procedure.

As shown in FIG. 13, treatment with ch425 alone at a dose of 5 mg/kg had little or no effect on tumor growth. 317-4B6 at a dose of 1 mg/kg showed a trend toward delayed tumor growth without statistical significance (P>0,05). However, combined treatment with ch425 and 317-4B6 showed significantly synergistic effects, inhibiting tumor growth by more than 60% versus the vehicle-treated group.

The results indicated that combination therapy of ch425 with 317-4B6 could activate human immune cells to inhibit tumor growth in a mouse in vivo cancer model, which was consistent with the in vitro data described in Example 11.

What is claimed is:

1. An antibody capable of binding to human Tim-3, comprising:
(a) a heavy chain variable region (VH) comprising one, two or three CDR amino acid sequences selected form SEQ ID NOs 3, 4, 5, or 26 or variants thereof comprising one or more conservative substitutions; and/or
(b) a light chain variable region (VL) comprising one, two or three CDR amino acid sequences selected form SEQ ID NOs 6, 7, 8, or 27 or variants thereof comprising one or more conservative substitutions.

2. The antibody of claim 1, wherein the antibody comprises:
(a) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 8 or variants thereof comprising one or more conservative substitutions;
(b) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 26 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 8 or variants thereof comprising one or more conservative substitutions;
(c) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 4 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions; or
(d) a heavy chain variable region (VH) comprising a VH-CDR1 amino acid sequence of SEQ ID NO 3 or variants thereof comprising one or more conservative substitutions, a VH-CDR2 amino acid sequence of SEQ ID NO 26 or variants thereof comprising one or more conservative substitutions and a VH-CDR3 amino acid sequence of SEQ ID NO 5 or variants thereof comprising one or more conservative substitutions; and a light chain variable region (VL) comprising a VL-CDR1 amino acid sequence of SEQ ID NO 6 or variants thereof comprising one or more conservative substitutions, a VL-CDR2 amino acid sequence of SEQ ID NO 7 or variants thereof comprising one or more conservative substitutions and a VL-CDR3 amino acid sequence of SEQ ID NO 27 or variants thereof comprising one or more conservative substitutions.

3. The antibody of claim 1, wherein the antibody comprises:
(a) a heavy chain variable region (VH) comprising a VH-CDR1 amino acid sequence of SEQ ID NO 3, a VH-CDR2 amino acid sequence of SEQ ID NO 4 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 8; or
(b) a heavy chain variable region (VH) comprising a VH-CDR1 amino acid sequence of SEQ ID NO 3, a VH-CDR2 amino acid sequence of SEQ ID NO 26 and a VH-CDR3 amino acid sequence of SEQ ID NO 5; and a light chain variable region (VL) comprising a VL-CDR1 amino acid sequence of SEQ ID NO 6, a VL-CDR2 amino acid sequence of SEQ ID NO 7 and a VL-CDR3 amino acid sequence of SEQ ID NO 27.

4. The antibody of claim 1, wherein the antibody is a humanized antibody molecule.

5. The antibody of claim 1, wherein the antibody comprises a heavy chain variable domain having at least 95%, 96%, 97%, 98%, 99%, or 100% sequence identity with the amino acid sequence of SEQ ID NOs 9, 17, 28 or 40.

6. The antibody of claim 1, wherein the antibody comprises a heavy chain variable domain having at least 95%, 96%, 97%, 98%, 99%, or 100% sequence identity with the amino acid sequence of SEQ ID NOs 9, 17 or 28.

7. The antibody of claim 1, wherein the antibody comprises a light chain variable domain having at least 95%, 96%, 97%, 98%, 99%, or 100% sequence identity with the amino acid sequence of SEQ ID NO 11, 19, 30 or 36.

8. The antibody of claim 1, wherein the antibody comprises:
(a) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 11;
(b) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 19;
(c) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99% or 100% sequence identity with the amino acid sequence of SEQ ID NO 30;
(d) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 36;
(e) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 11;
(f) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 19;
(g) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 30;
(h) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 36;
(i) a heavy chain variable domain having at least 95%, 96%, 97%, 98%, 99%, or 100% sequence identity with the amino acid sequence of SEQ ID NO 28, and a light chain variable domain having at least 95%, 96%, 97%, 98%, 99%, or 100% sequence identity with the amino acid sequence of SEQ ID NO 11;
(j) a heavy chain variable domain having at least 95%, 96%, 97%, 98%, 99%, or 100% sequence identity with the amino acid sequence of SEQ ID NO 28, and a light chain variable domain having at least 95%, 96%, 97%, 98%, 99%, or 100% sequence identity with the amino acid sequence of SEQ ID NO 19;
(k) a heavy chain variable domain having at least 95%, 96%, 97%, 98%, 99%, or 100% sequence identity with the amino acid sequence of SEQ ID NO 28, and a light chain variable domain having at least 95%, 96%, 97%, 98%, 99%, or 100% sequence identity with the amino acid sequence of SEQ ID NO 30;
(l) a heavy chain variable domain having at least 95%, 96%, 97%, 98%, 99%, or 100% sequence identity with the amino acid sequence of SEQ ID NO 28, and a light chain variable domain having at least 95%, 96%, 97%, 98%, 99%, or 100% sequence identity with the amino acid sequence of SEQ ID NO 36;
(m) a heavy chain variable domain having at least 95%, 96%, 97%, 98%, 99%, or 100% sequence identity with the amino acid sequence of SEQ ID NO 40, and a light chain variable domain having at least 95%, 96%, 97%, 98%, 99%, or 100% sequence identity with the amino acid sequence of SEQ ID NO 11;
(n) a heavy chain variable domain having at least 95%, 96%, 97%, 98%, 99%, or 100% sequence identity with the amino acid sequence of SEQ ID NO 40, and a light chain variable domain having at least 95%, 96%, 97%, 98%, 99%, or 100% sequence identity with the amino acid sequence of SEQ ID NO 19;
(o) a heavy chain variable domain having at least 95%, 96%, 97%, 98%, 99%, or 100% sequence identity with the amino acid sequence of SEQ ID NO 40, and a light chain variable domain having at least 95%, 96%, 97%, 98%, 99%, or 100% sequence identity with the amino acid sequence of SEQ ID NO 30; or
(p) a heavy chain variable domain having at least 95%, 96%, 97%, 98%, 99%, or 100% sequence identity with the amino acid sequence of SEQ ID NO 40, and a light chain variable domain having at least 95%, 96%, 97%, 98%, 99%, or 100% sequence identity with the amino acid sequence of SEQ ID NO 36.

9. The antibody of claim 1, wherein the antibody comprises:
(a) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 11;
(b) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 19;
(c) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 30;
(d) a heavy chain variable domain having at least 95%, 96%, 97%, 98%, 99%, or 100% sequence identity with the amino acid sequence of SEQ ID NO 9, and a light chain variable domain having at least 95%, 96%, 97%, 98%, 99%, or 100% sequence identity with the amino acid sequence of SEQ ID NO 36;
(e) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 11;
(f) a heavy chain variable domain having at least 95%, 96%, 97%, 98%, 99%, or 100% sequence identity with the amino acid sequence of SEQ ID NO 17, and a light chain variable domain having at least 95%, 96%, 97%, 98%, 99%, or 100% sequence identity with the amino acid sequence of SEQ ID NO 19;
(g) a heavy chain variable domain having at least 95%, 96%, 97%, 98%, 99%, or 100% sequence identity with the amino acid sequence of SEQ ID NO 17, and a light chain variable domain having at least 9595%, 96%, 97%, 98%, 99%, or 100% sequence identity with the amino acid sequence of SEQ ID NO 30;
(h) a heavy chain variable domain having at least 9595%, 96%, 97%, 98%, 99%, or 100% sequence identity with the amino acid sequence of SEQ ID NO 17, and a light chain variable domain having at least 9595%, 96%, 97%, 98%, 99%, or 100% sequence identity with the amino acid sequence of SEQ ID NO 36;
(i) a heavy chain variable domain having at least 9595%, 96%, 97%, 98%, 99%, or 100% sequence identity with the amino acid sequence of SEQ ID NO 28, and a light chain variable domain having at least 9595%, 96%, 97%, 98%, 99%, or 100% sequence identity with the amino acid sequence of SEQ ID NO 11;
(j) a heavy chain variable domain having at least 95%, 96%, 97%, 98%, 99%, or 100% sequence identity with the amino acid sequence of SEQ ID NO 28, and a light chain variable domain having at least 95%, 96%, 97%, 98%, 99%, or 100% sequence identity with the amino acid sequence of SEQ ID NO 19;
(k) a heavy chain variable domain having at least 9595%, 96%, 97%, 98%, 99%, or 100% sequence identity with the amino acid sequence of SEQ ID NO 28, and a light chain variable domain having at least 9595%, 96%, 97%, 98%, 99%, or 100% sequence identity with the amino acid sequence of SEQ ID NO 30; or
(l) a heavy chain variable domain having at least 9595%, 96%, 97%, 98%, 99%, or 100% sequence identity with the amino acid sequence of SEQ ID NO 28, and a light chain variable domain having at least 9595%, 96%, 97%, 98%, 99%, or 100% sequence identity with the amino acid sequence of SEQ ID NO 36.

10. The antibody of claim 1, wherein the antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30;
(d) a heavy chain variable domain comprising the amino acid sequence of SEQ ID NO 28, and a light chain variable domain comprising the amino acid sequence of SEQ ID NO 36;
(e) a heavy chain variable domain comprising the amino acid sequence of SEQ ID NO 40, and a light chain variable domain comprising the amino acid sequence of SEQ ID NO 36.

11. The antibody of claim 1, wherein the antibody comprises:
(a) a heavy chain variable domain comprising the amino acid sequence of SEQ ID NO 9, and a light chain variable domain comprising the amino acid sequence of SEQ ID NO 11;
(b) a heavy chain variable domain comprising the amino acid sequence of SEQ ID NO 17, and a light chain variable domain comprising the amino acid sequence of SEQ ID NO 19;
(c) a heavy chain variable domain comprising the amino acid sequence of SEQ ID NO 28, and a light chain variable domain comprising the amino acid sequence of SEQ ID NO 30; or
(d) a heavy chain variable domain comprising the amino acid sequence of SEQ ID NO 28, and a light chain variable domain comprising the amino acid sequence of SEQ ID NO 36.

12. The antibody of claim 1, wherein the antibody comprises a heavy chain having at least 95%, 96%, 97%, 98%, 99%, or 100% sequence identity with the amino acid sequence of SEQ ID NO 13, 22 or 32.

13. The antibody of claim 1, wherein the antibody comprises a light chain having at least 995%, 96%, 97%, 98%, 99%, or 100% sequence identity with the amino acid sequence of SEQ ID NO 15, 24, 34 or 38.

14. The antibody of claim 1, wherein the antibody comprises:
(a) a heavy chain having at least 95%, 96%, 97%, 98%, 99%, or 100% sequence identity with the amino acid sequence of SEQ ID NO 13, and a light chain having at least 95%, 96%, 97%, 98%, 99%, or 100% sequence identity with the amino acid sequence of SEQ ID NO 15;
(b) a heavy chain having at least 95%, 96%, 97%, 98%, 99%, or 100% sequence identity with the amino acid sequence of SEQ ID NO 13, and a light chain having at least 95%, 96%, 97%, 98%, 99%, or 100% sequence identity with the amino acid sequence of SEQ ID NO 24;
(c) a heavy chain having at least 95%, 96%, 97%, 98%, 99%, or 100% sequence identity with the amino acid sequence of SEQ ID NO 13, and a light chain having at least 95%, 96%, 97%, 98%, 99%, or 100% sequence identity with the amino acid sequence of SEQ ID NO 34;
(d) a heavy chain having at least 95%, 96%, 97%, 98%, 99%, or 100% sequence identity with the amino acid sequence of SEQ ID NO 13, and a light chain having at least 95%, 96%, 97%, 98%, 99%, or 100% sequence identity with the amino acid sequence of SEQ ID NO 38;
(e) a heavy chain having at least 9595%, 96%, 97%, 98%, 99%, or 100% sequence identity with the amino acid sequence of SEQ ID NO 22, and a light chain having at least 9595%, 96%, 97%, 98%, 99%, or 100% sequence identity with the amino acid sequence of SEQ ID NO 15;
(f) a heavy chain having at least 9595%, 96%, 97%, 98%, 99%, or 100% sequence identity with the amino acid sequence of SEQ ID NO 22, and a light chain having at least 9595%, 96%, 97%, 98%, 99%, or 100% sequence identity with the amino acid sequence of SEQ ID NO 24;
(g) a heavy chain having at least 95%, 96%, 97%, 98%, 99%, or 100% sequence identity with the amino acid sequence of SEQ ID NO 22, and a light chain having at least 95%, 96%, 97%, 98%, 99%, or 100% sequence identity with the amino acid sequence of SEQ ID NO 34;
(h) a heavy chain having at least 95%, 96%, 97%, 98%, 99%, or 100% sequence identity with the amino acid sequence of SEQ ID NO 22, and a light chain having at least 9595%, 96%, 97%, 98%, 99%, or 100% sequence identity with the amino acid sequence of SEQ ID NO 38;
(i) a heavy chain having at least 95%, 96%, 97%, 98%, 99%, or 100% sequence identity with the amino acid sequence of SEQ ID NO 32, and a light chain having at least 95%, 96%, 97%, 98%, 99%, or 100% sequence identity with the amino acid sequence of SEQ ID NO 15;
(j) a heavy chain having at least 95%, 96%, 97%, 98%, 99%, or 100% sequence identity with the amino acid sequence of SEQ ID NO 32, and a light chain having at least 95%, 96%, 97%, 98%, 99%, or 100% sequence identity with the amino acid sequence of SEQ ID NO 24;
(k) a heavy chain having at least 95%, 96%, 97%, 98%, 99%, or 100% sequence identity with the amino acid sequence of SEQ ID NO 32, and a light chain having at least 95%, 96%, 97%, 98%, 99%, or 100% sequence identity with the amino acid sequence of SEQ ID NO 34; or
(l) a heavy chain having at least 95%, 96%, 97%, 98%, 99%, or 100% sequence identity with the amino acid sequence of SEQ ID NO 32, and a light chain having at least 9595%, 96%, 97%, 98%, 99%, or 100% sequence identity with the amino acid sequence of SEQ ID NO 38.

15. The antibody of claim 1, wherein the antibody comprises:
(a) a heavy chain comprising the amino acid sequence of SEQ ID NO 13, and a light chain comprising the amino acid sequence of SEQ ID NO 15;
(b) a heavy chain comprising the amino acid sequence of SEQ ID NO 22, and a light chain comprising the amino acid sequence of SEQ ID NO 24;
(c) a heavy chain comprising the amino acid sequence of SEQ ID NO 32, and a light chain comprising the amino acid sequence of SEQ ID NO 34; or
(d) a heavy chain comprising the amino acid sequence of SEQ ID NO 32, and a light chain comprising the amino acid sequence of SEQ ID NO 38.

16. The antibody of any one of claims 1-15, wherein the antibody comprises one or more of:
(a) a light chain with an Aspartic acid to Glutamic acid mutation at position 1 of SEQ ID NO 24;
(b) a light chain with a Leucine to Methionine mutation at position 4 of SEQ ID NO 24;
(c) a light chain with a Valine to Isoleucine mutation at position 62 of SEQ ID NO 24;
(d) a light chain with a Aspartic acid to Glutamic acid mutation at position 74 of SEQ ID NO 24;
(e) a light chain with a Methionine to Leucine mutation at position 96 of SEQ :D NO 24;
(f) a heavy chain with a Phenylalanine to Tyrosine mutation at position 59 of SEQ ID NO 22;
(h) (g) a heavy chain with a Proline to Valine mutation at position 60 of SEQ ID NO 22; a heavy chain with a Serine to Threonine mutation at position 77 of SEQ ID NO 22; or
(i) a heavy chain with a Cysteine to Leucine mutation at position 78 of SEQ ID NO 22.

17. The antibody of claim 1, wherein the antibody is a Fab, F(ab′)2, Fv, or a single chain Fv (ScFv).

18. The antibody of claim 1, wherein the antibody comprises a heavy chain constant region of the subclass of IgC1, IgG2, IgG3, or IgG4 or a variant thereof, and a light chain constant region of the type of kappa or lambda or a variant thereof.

19. The antibody of claim 18, wherein the antibody comprises a variant heavy chain constant region of the subclass of IgG1, IgG2, IgG3, or IgG4, wherein the variant heavy chain constant region provides a reduced or eliminated effector function.

20. The antibody of claim 19, wherein the effector function is antibody-dependent cell-mediated cytotoxicity (ADCC) or complement-dependent cytotoxicity (CDC).

21. The antibody of claim 18, wherein the antibody comprises a heavy chain constant region of human IgG1 or a variant thereof.

22. The antibody of claim 21, wherein the variant heavy chain constant region of human IgG1 comprises one or more mutations selected from a group consisting of E₂₃₃P, L₂₃₄A, L₂₃₅A, L₂₃₆Δ and P₃₂₉A.

23. The antibody of claim 22, wherein the variant human IgG1 heavy chain constant region comprising the amino sequence of SEQ ID NO 21, and a human kappa light chain constant religion.

24. A pharmaceutical composition, comprising the antibody of any one of claims 1-23, and a pharmaceutically acceptable excipient.

25. A method of stimulating an immune response in a subject, comprising administrating to a subject in need thereof an antibody of claim 1 in an amount effective to stimulate the immune respond.

26. A method for treating a cancer or a tumor, comprising administrating to a subject in need thereof the antibody of claim 1 in an amount effective to treat the cancer or tumor.

27. The method of claim 26, wherein the cancer is selected from a lung cancer, a liver cancer, a stomach cancer, a cervical cancer, a melanoma, a renal cancer, a breast cancer, a colorectal cancer, a leukemia, a lymphoma, an ovarian cancer, a head and neck cancer or a metastatic lesion of the cancer.

28. The method of claim 26, wherein the antibody is administrated in combination with a second therapeutic agent or procedure, wherein the second therapeutic agent or procedure is selected from a chemotherapy, a targeted therapy, an oncolytic drug, a cytotoxic agent, an immune-based therapy, a cytokine, a surgical procedure, a radiation procedure, an activator of a costimulatory molecule, an inhibitor of an inhibitory molecule, a vaccine, or a cellular immunotherapy.

29. The method of claim 26, wherein the antibody is administered in combination with an inhibitor of an immune checkpoint molecule selected from PD-1, PD-L1, PD-L2, CTLA-4, LAG-3, CEACAM-1, CEACAM-5, VISTA, BTLA, TIGIT, LAIR1, CD160, 2B4 or TGFR.

30. The method of claim 26, wherein the antibody is administered in combination with an anti-PD-1 mAb 317-4B6, or 317-4B6/IgG4mt10.

31. A method of treating an infectious disease, comprising administering to a subject in need thereof an antibody of claim 1 in an amount effective to treat the infectious disease.

32. The method of claim 31, wherein the infectious disease is a chronic viral infection, selected from HIV infection and HCV infection.

33. Use of the anti-Tim-3 antibody molecules of any one of claim 1-23 in manufacturing medicine for treating a cancer, a tumor or an infectious disease.


SEQUENCE LISTING (43 sequences)

SEQ ID NO: 1 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - protein, 202 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIG

SEQ ID NO: 3 - protein, 5 aa, Mus musculus
RYAMS

SEQ ID NO: 4 - protein, 16 aa, Mus musculus
AISSGGSLYFPDSVKG

SEQ ID NO: 5 - protein, 11 aa, Mus musculus
GREADGGYFDY

SEQ ID NO: 6 - protein, 15 aa, Mus musculus
RASESVEYYGTSLMQ

SEQ ID NO: 7 - protein, 7 aa, Mus musculus
AASNVES

SEQ ID NO: 8 - protein, 9 aa, Mus musculus
QQSMKVPLT

SEQ ID NO: 9 - protein, 119 aa, Mus musculus
EVKLVESGGGLVKPGGSLKLSCAASGFTFSRYAMSWVRQIPEKRLEWVAAISSGGSLYFPDSVKGRFTISRDNARNICYLQMNSLRSDDTAMYYCARGREADGGYFDYWGQGTTLTVSS

SEQ ID NO: 10 - DNA, 357 bp, Mus musculus
  source 1..357: mol_type = other DNA
gaagtgaagctggtggagtctgggggaggcttagtgaagcctggagggtccctgaaactctcctgtgcagcctctggattcactttcagtaggtatgccatgtcttgggttcgccagattccagagaagaggctggagtgggtcgcagccattagtagtggtggtagtttatactttccagacagtgtgaagggccgattcaccatctccagagataatgccaggaacatctgctacctgcaaatgaacagtctgaggtctgacgacacggccatgtattactgtgcaagaggccgggaggccgacggaggctactttgactactggggccaaggcaccactctcacagtctcctca

SEQ ID NO: 11 - protein, 111 aa, Mus musculus
DIVLTQSPASLAVSLGQRATISCRASESVEYYGTSLMQWYQQKPGQPPKLLIYAASNVESGVPARFSGSGSGTDFSLNIHPVEEDDIAMYFCQQSMKVPLTFGAGTKLELK

SEQ ID NO: 12 - DNA, 333 bp, Mus musculus
  source 1..333: mol_type = other DNA
gacattgtgctcacccaatctccagcttctttggctgtgtctctagggcagagagccaccatctcctgcagagccagtgaaagtgttgaatattatggcacaagtttaatgcagtggtaccaacagaaaccaggacaaccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaaagtatgaaggttcctctcacgttcggtgctgggaccaagctggagctgaaa

SEQ ID NO: 13 - protein, 448 aa, synthetic construct
  REGION 1..448: note = ch425 heavy chain pro
EVKLVESGGGLVKPGGSLKLSCAASGFTFSRYAMSWVRQIPEKRLEWVAAISSGGSLYFPDSVKGRFTISRDNARNICYLQMNSLRSDDTAMYYCARGREADGGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1347 bp, synthetic construct
  misc_feature 1..1347: note = ch425 heavy chain DNA
  source 1..1347: mol_type = other DNA
gaagtgaagctggtggagtctgggggaggcttagtgaagcctggagggtccctgaaactctcctgtgcagcctctggattcactttcagtaggtatgccatgtcttgggttcgccagattccagagaagaggctggagtgggtcgcagccattagtagtggtggtagtttatactttccagacagtgtgaagggccgattcaccatctccagagataatgccaggaacatctgctacctgcaaatgaacagtctgaggtctgacgacacggccatgtattactgtgcaagaggccgggaggccgacggaggctactttgactactggggccaaggcaccactctcacagtctcctcagcctccaccaagggcccatcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctcctgctgccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcgccgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 218 aa, synthetic construct
  REGION 1..218: note = ch425 light chain pro
DIVLTQSPASLAVSLGQRATISCRASESVEYYGTSLMQWYQQKPGQPPKLLIYAASNVESGVPARFSGSGSGTDFSLNIHPVEEDDIAMYFCQQSMKVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 657 bp, synthetic construct
  misc_feature 1..657: note = ch425 light chain DNA
  source 1..657: mol_type = other DNA
gacattgtgctcacccaatctccagcttctttggctgtgtctctagggcagagagccaccatctcctgcagagccagtgaaagtgttgaatattatggcacaagtttaatgcagtggtaccaacagaaaccaggacaaccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaaagtatgaaggttcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 17 - protein, 119 aa, synthetic construct
  REGION 1..119: note = hu425-1-1 VH pro
EVQLVESGGGLVQPGGSLRLSCAASGFTFSRYAMSWVRQAPGKGLEWVAAISSGGSLYFPDSVKGRFTISRDNAKNSCYLQMNSLRAEDTAVYYCARGREADGGYFDYWGQGTLVTVSS

SEQ ID NO: 18 - DNA, 357 bp, synthetic construct
  misc_feature 1..357: note = hu425-1-1 VH DNA
  source 1..357: mol_type = other DNA
gaagtgcagctggtcgaatcagggggggggctggtgcagcctggaggcagcctgagactgtcctgcgccgcttctggcttcacctttagcagatacgccatgtcctgggtgcggcaggctcctggcaagggactggagtgggtggccgctatcagctccggcggctccctgtacttccccgattccgtgaagggccggttcaccatcagcagggacaacgccaagaacagctgctatctgcagatgaactctctgagggccgaggatacagccgtgtactattgcgctcggggcagagaagcagatggcggctacttcgactattggggccagggcaccctggtgacagtgtctagc

SEQ ID NO: 19 - protein, 111 aa, synthetic construct
  REGION 1..111: note = hu425-1-1 VK pro
DIVLTQSPATLSVSPGERATLSCRASESVEYYGTSLMQWYQQKPGQAPRLLIYAASNVESGVPARFSGSGSGTDFTLTISSLQSEDFAVYYCQQSMKVPLTFGGGTKVEIK

SEQ ID NO: 20 - DNA, 333 bp, synthetic construct
  misc_feature 1..333: note = hu425-1-1 VK DNA
  source 1..333: mol_type = other DNA
gacatcgtcctgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcgtcccagcacgcttcagtggctcagggagcggaacagactttaccctgacaattagctccctgcagagtgaagatttcgccgtgtactattgccagcagagcatgaaggtccccctgacatttggcgggggaactaaggtggagatcaaa

SEQ ID NO: 21 - protein, 329 aa, synthetic construct
  REGION 1..329: note = IgG1mf pro
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 448 aa, synthetic construct
  REGION 1..448: note = hu425-1-1 heavy chain pro
EVQLVESGGGLVQPGGSLRLSCAASGFTFSRYAMSWVRQAPGKGLEWVAAISSGGSLYFPDSVKGRFTISRDNAKNSCYLQMNSLRAEDTAVYYCARGREADGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1347 bp, synthetic construct
  misc_feature 1..1347: note = hu425-1-1 heavy chain DNA
  source 1..1347: mol_type = other DNA
gaagtgcagctggtcgaatcagggggggggctggtgcagcctggaggcagcctgagactgtcctgcgccgcttctggcttcacctttagcagatacgccatgtcctgggtgcggcaggctcctggcaagggactggagtgggtggccgctatcagctccggcggctccctgtacttccccgattccgtgaagggccggttcaccatcagcagggacaacgccaagaacagctgctatctgcagatgaactctctgagggccgaggatacagccgtgtactattgcgctcggggcagagaagcagatggcggctacttcgactattggggccagggcaccctggtgacagtgtctagcgctagcaccaaaggccccagcgtgtttcctctggctccatcctctaaatccacctctggcggcacagccgctctgggctgtctggtgaaggattacttcccagagcccgtgacagtgtcttggaacagcggcgccctgacctccggcgtgcacacatttcctgctgtgctgcagagctccggcctgtacagcctgtctagcgtggtgaccgtgccatcctctagcctgggcacccagacatatatctgcaacgtgaatcacaagcccagcaatacaaaggtggataagaaggtggagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggatgagctgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaatga

SEQ ID NO: 24 - protein, 218 aa, synthetic construct
  REGION 1..218: note = hu425-1-1 light chain pro
DIVLTQSPATLSVSPGERATLSCRASESVEYYGTSLMQWYQQKPGQAPRLLIYAASNVESGVPARFSGSGSGTDFTLTISSLQSEDFAVYYCQQSMKVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 657 bp, synthetic construct
  misc_feature 1..657: note = hu425-1-1 light chain DNA
  source 1..657: mol_type = other DNA
gacatcgtcctgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcgtcccagcacgcttcagtggctcagggagcggaacagactttaccctgacaattagctccctgcagagtgaagatttcgccgtgtactattgccagcagagcatgaaggtccccctgacatttggcgggggaactaaggtggagatcaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgctaa

SEQ ID NO: 26 - protein, 16 aa, synthetic construct
  REGION 1..16: note = hu425-2-2 HCDR2
AISSGGSLYYPDSVKG

SEQ ID NO: 27 - protein, 9 aa, synthetic construct
  REGION 1..9: note = hu425-2-2 LCDR3
QQSLKVPLT

SEQ ID NO: 28 - protein, 119 aa, synthetic construct
  REGION 1..119: note = hu425-2-2 VH pro
EVQLVESGGGLVQPGGSLRLSCAASGFTFSRYAMSWVRQAPGKGLEWVAAISSGGSLYYPDSVKGRFTISRDNAKNTLYLQMNSLRAEDTAVYYCARGREADGGYFDYWGQGTLVTVSS

SEQ ID NO: 29 - DNA, 357 bp, synthetic construct
  misc_feature 1..357: note = hu425-2-2 VH DNA
  source 1..357: mol_type = other DNA
gaagtgcagctggtcgaatcagggggggggctggtgcagcctggaggcagcctgagactgtcctgcgccgcttctggcttcacctttagcagatacgccatgtcctgggtgcggcaggctcctggcaagggactggagtgggtggccgctatcagctccggcggctccctgtactatcccgattccgtgaagggccggttcaccatcagcagggacaacgccaagaacacactgtatctgcagatgaactctctgagggccgaggatacagccgtgtactattgcgctcggggcagagaagcagatggcggctacttcgactattggggccagggcaccctggtgacagtgtctagc

SEQ ID NO: 30 - protein, 111 aa, synthetic construct
  REGION 1..111: note = hu425-2-2 VK pro
EIVMTQSPATLSVSPGERATLSCRASESVEYYGTSLMQWYQQKPGQAPRLLIYAASNVESGIPARFSGSGSGTEFTLTISSLQSEDFAVYYCQQSLKVPLTFGGGTKVEIK

SEQ ID NO: 31 - DNA, 333 bp, synthetic construct
  misc_feature 1..333: note = hu425-2-2 VK DNA
  source 1..333: mol_type = other DNA
gagatcgtcatgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcatcccagcacgcttcagtggctcagggagcggaacagagtttaccctgacaattagctccctgcagagtgaagatttcgccgtgtactattgccagcagagcctgaaggtccccctgacatttggcgggggaactaaggtggagatcaaa

SEQ ID NO: 32 - protein, 448 aa, synthetic construct
  REGION 1..448: note = hu425-2-2 heavy chain pro
EVQLVESGGGLVQPGGSLRLSCAASGFTFSRYAMSWVRQAPGKGLEWVAAISSGGSLYYPDSVKGRFTISRDNAKNTLYLQMNSLRAEDTAVYYCARGREADGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1347 bp, synthetic construct
  misc_feature 1..1347: note = hu425-2-2 heavy chain DNA
  source 1..1347: mol_type = other DNA
gaagtgcagctggtcgaatcagggggggggctggtgcagcctggaggcagcctgagactgtcctgcgccgcttctggcttcacctttagcagatacgccatgtcctgggtgcggcaggctcctggcaagggactggagtgggtggccgctatcagctccggcggctccctgtactatcccgattccgtgaagggccggttcaccatcagcagggacaacgccaagaacacactgtatctgcagatgaactctctgagggccgaggatacagccgtgtactattgcgctcggggcagagaagcagatggcggctacttcgactattggggccagggcaccctggtgacagtgtctagcgctagcaccaaaggccccagcgtgtttcctctggctccatcctctaaatccacctctggcggcacagccgctctgggctgtctggtgaaggattacttcccagagcccgtgacagtgtcttggaacagcggcgccctgacctccggcgtgcacacatttcctgctgtgctgcagagctccggcctgtacagcctgtctagcgtggtgaccgtgccatcctctagcctgggcacccagacatatatctgcaacgtgaatcacaagcccagcaatacaaaggtggataagaaggtggagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggatgagctgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaatga

SEQ ID NO: 34 - protein, 218 aa, synthetic construct
  REGION 1..218: note = hu425-2-2 light chain pro
EIVMTQSPATLSVSPGERATLSCRASESVEYYGTSLMQWYQQKPGQAPRLLIYAASNVESGIPARFSGSGSGTEFTLTISSLQSEDFAVYYCQQSLKVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 657 bp, synthetic construct
  misc_feature 1..657: note = hu425-2-2 light chain DNA
  source 1..657: mol_type = other DNA
gagatcgtcatgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcatcccagcacgcttcagtggctcagggagcggaacagagtttaccctgacaattagctccctgcagagtgaagatttcgccgtgtactattgccagcagagcctgaaggtccccctgacatttggcgggggaactaaggtggagatcaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgctaa

SEQ ID NO: 36 - protein, 111 aa, synthetic construct
  REGION 1..111: note = hu425-2-3b VK pro
EIVLTQSPATLSVSPGERATLSCRASESVEYYGTSLMQWYQQKPGQAPRLLIYAASNVESGIPARFSGSGSGTEFTLTISSLQSEDFAVYYCQQSLKVPLTFGGGTKVEIK

SEQ ID NO: 37 - DNA, 333 bp, synthetic construct
  misc_feature 1..333: note = hu425-2-3b VK DNA
  source 1..333: mol_type = other DNA
gagatcgtcctgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcatcccagcacgcttcagtggctcagggagcggaacagagtttaccctgacaattagctccctgcagagtgaagatttcgccgtgtactattgccagcagagcctgaaggtccccctgacatttggcgggggaactaaggtggagatcaaa

SEQ ID NO: 38 - protein, 218 aa, synthetic construct
  REGION 1..218: note = hu425-2-3b light chain pro
EIVLTQSPATLSVSPGERATLSCRASESVEYYGTSLMQWYQQKPGQAPRLLIYAASNVESGIPARFSGSGSGTEFTLTISSLQSEDFAVYYCQQSLKVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 657 bp, synthetic construct
  misc_feature 1..657: note = hu425-2-3b light chain DNA
  source 1..657: mol_type = other DNA
gagatcgtcctgactcagtcccctgccactctgtcagtgagcccaggagagcgagctaccctgtcctgcagagcatccgagtctgtcgaatactatggcacctctctgatgcagtggtaccagcagaagccagggcaggctcccaggctgctgatctatgccgcttctaacgtggagagtggcatcccagcacgcttcagtggctcagggagcggaacagagtttaccctgacaattagctccctgcagagtgaagatttcgccgtgtactattgccagcagagcctgaaggtccccctgacatttggcgggggaactaaggtggagatcaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgctaa

SEQ ID NO: 40 - protein, 118 aa, synthetic construct
  REGION 1..118: note = artificial sequences
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 41 - protein, 111 aa, synthetic construct
  REGION 1..111: note = artificial sequences
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 42 - protein, 114 aa, synthetic construct
  REGION 1..114: note = artificial sequences
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 43 - protein, 108 aa, synthetic construct
  REGION 1..108: note = artificial sequences
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR